(19) United States
(12) Patent Application Publication
(10) Pub. No.: US 2026/0285944 A1
(43) Pub. Date: Sep. 24, 2026

(54) STABLE HEMOGLOBIN PROTEIN COMPOSITION

(71) Applicant: Prolong Pharmaceuticals, LLC, South Plainfield, NJ (US)

(72) Inventors: Shaunak G. Pandya, Fogelsville, PA (US); Ronald Jubin, Watchung, NJ (US)

(21) Appl. No.: 19/684,471

(22) Filed: May 21, 2026

Related U.S. Application Data
(63) Continuation of application No. 19/121,002, filed on Oct. 13, 2023.
(60) Provisional application No. 63/416,434, filed on Oct. 14, 2022.

(51) Int. Cl. C07K 14/805; A61K 38/00; C07K 1/34; G01N 33/72

(52) U.S. Cl. CPC C07K 14/805; C07K 1/34; G01N 33/721; A61K 38/00; G01N 2333/805

(57) ABSTRACT

The present invention relates to a hemoglobin protein composition comprising biologically active hemoglobin and less than 2% methemoglobin (metHb) wherein the metHb is maintained below 2% after reoxygenation of hemoglobin. Furthermore, hemoglobin protein composition comprising biologically active hemoglobin and less than 25% of charge variants of total oxygenated hemoglobin. Moreover, the present invention also provides an effective concentration of antioxidant more than 5 mM used during heat treatment. The present invention provides pharmaceutically stable composition of hemoglobin. Furthermore, the present invention also provides a process for reducing and/or controlling the formation of metHb, charge variants and inactivation of viral and/or prion during manufacturing of oxygenated hemoglobin. In addition, the invention provides therapeutic use of oxygenated hemoglobin.

### CROSS-REFERENCE TO RELATED APPLICATIONS

This application is a continuation of U.S. patent application Ser. No. 19/121,002, filed on Apr. 14, 2025, which is a U.S. national stage patent application of International Application No. PCT/US2023/076908, filed on Oct. 13, 2023, which claims the benefit of and priority to U.S. Provisional Application No. 63/416,434, filed on Oct. 14, 2022, each of which is incorporated herein by reference in its entirety.

### FIELD OF INVENTION

The present invention relates to a hemoglobin protein composition (e.g., a stable hemoglobin protein composition) comprising biologically active hemoglobin and less than 2% methemoglobin (metHb) wherein the metHb is maintained below 2% after reoxygenation of hemoglobin.

Furthermore, hemoglobin protein composition comprising biologically active hemoglobin and less than 25% of charge variants of total oxygenated hemoglobin. Moreover, the present invention also provides an effective concentration of antioxidant more than 5 mM used during heat treatment. The present invention provides pharmaceutically stable composition of hemoglobin. Furthermore, the present invention also provides a process for reducing and/or controlling the formation of metHb, charge variants and inactivation of viral and/or prion during manufacturing of oxygenated hemoglobin. In addition, the invention provides therapeutic use of oxygenated hemoglobin.

### BACKGROUND OF THE INVENTION

The development of hemoglobin-based oxygen carriers (HBOC) has focused on oxygen delivery for use in medical therapies such as transfusions. HBOCs have been shown to prevent or treat hypoxia resulting from blood loss (e.g., from acute hemorrhage or during surgical operations), from anemia (e.g., pernicious anemia or sickle cell anemia), or from shock (e.g., hypovolemic shock, anaphylactic shock, septic shock or allergic shock).

Existing hemoglobin-based oxygen carriers include synthesized hemoglobin analogues, liposome-encapsulated hemoglobin, chemically modified hemoglobin, and hemoglobin-based oxygen carriers in which the hemoglobin molecules are cross-linked.

Hemoglobin is highly susceptible to oxidation in presence of oxygen which leads to the formation of oxidized hemoglobin known as Methemoglobin. Methemoglobin is a hemoglobin, in which the iron in the heme group is in the Fe³⁺ (ferric) state, not the Fe²⁺ (ferrous) of normal hemoglobin.

Sometimes, it is also referred to as ferrihemoglobin. MetHb cannot bind oxygen, which means it cannot carry oxygen to tissues. It is bluish chocolate-brown in color. In human blood a trace amount of methemoglobin is normally produced spontaneously, but when present in excess the blood becomes abnormally dark bluish brown. The NADH-dependent enzyme methemoglobin reductase (a type of diaphorase) is responsible for converting methemoglobin back to hemoglobin.

Normally one to two percent of a person's hemoglobin is methemoglobin; higher amounts can be genetic or caused by exposure to various chemicals and drugs resulting in the clinical condition of methemoglobinemia. High MetHb produces a functional anemia state promoting tissue hypoxia. Additionally extracellular MetHb has been shown to possess a Danger Associated Molecular Pattern (DAMP) potent inflammatory factor. This DAMP activity has been shown to include Complement and Toll-like Receptor 4 (TLR4) activation promoting macrophage activation, endothelial dysfunction and other activities. Many acute conditions including trauma, blood loss, infection may result in vascular injury/inflammation/dysfunction and transfusions of whole blood or early generation HBOCs were observed to promote morbidity and mortality rather than improving outcomes. These conditions are often exacerbated by elevated MetHb levels-either derived from the endogenous RBCs or transfused products containing hemoglobin.

Typical manufacturing steps for obtaining purified hemoglobin involves harsh purification and viral inactivation by high heat or chemicals. Unlike deoxygenated hemoglobin, which is stable at high temperatures, hemoglobin in the oxygenated state (HbO2) will denature and precipitate at high temperature and therefore, the final % HbO2 value is critical. All these processing steps results in an increase in auto-oxidation of hemoglobin and formation of methemoglobin. Importantly, this reduction in purity of hemoglobin often manifests during further processing i.e., reoxygenation step or a purification step where cysteine is removed. The prevention or controlling metHb levels during reoxygenated stage (oxyhemoglobin) is critical as in presence of oxygen, hemoglobin is more susceptible to oxidization. In addition, it was also found that charge variants are also critical to control during reoxygenated stage (oxyhemoglobin).

Consequently, there is a need in the art for effective control of metHb impurities and/or charge variants and preserving hemoglobin purity during manufacturing or purification process.

The present invention provides an effective control of hemoglobin purity and MetHb formation in oxyhemoglobin by using L-cysteine at a specific concentration or amount during manufacturing/purification stage. The present invention surprisingly found that the concentration of antioxidants greater than 5 mM can effectively control one or more metHb formation, charge variants and hemoglobin purity in oxyhemoglobin state (reoxygenated hemoglobin) during manufacturing/purification.

### SUMMARY OF INVENTION

In some embodiments, the present invention provides a hemoglobin composition comprising:
a) oxygenated hemoglobin; and
b) metHb less than 2% of total oxygenated hemoglobin.

In some embodiments, the present invention provides a composition of hemoglobin comprising:
a) oxygenated hemoglobin comprising main peak purity not less than 70%; and
b) one or more impurity selected from metHb, charge variants selected from acidic variants, basic variants and optionally virus particles and/or prions.

In some embodiments, the oxygenated hemoglobin comprising not more than 2% metHb of total oxygenated hemoglobin.

In some embodiments, the present invention provides a composition of hemoglobin comprising:
a) oxygenated hemoglobin comprising main peak purity not less than 70%;
b) one or more impurity selected from metHb, charge variants selected from acidic variants, basic variants and optionally virus particles and/or prions,
   wherein the oxygenated hemoglobin comprising not more than 2% metHb of total oxygenated hemoglobin.

In some embodiments, the present invention provides a composition of hemoglobin comprising:
a) oxygenated hemoglobin comprising main peak purity not less than 70% and one or more impurity comprising;
b) oxygenated hemoglobin comprising not more than 2% metHb;
c) oxygenated hemoglobin comprising less than 12% acidic variants of the main peak;
d) oxygenated hemoglobin comprising less than 11% basic variants of the main peak;
e) oxygenated hemoglobin comprising substantially free of virus particles; and
f) oxygenated hemoglobin comprising substantially free of prions.

In some embodiments, metHb is analyzed by co-oximetry and oxygenated hemoglobin main peak purity is analyzed by cIEF (capillary Iso Electric Focusing).

In some embodiments, the present invention provides a hemoglobin composition comprising:
a) oxygenated hemoglobin comprising main peak purity not less than 70%;
b) acidic variants are less than 12% of the main peak; and
c) basic variants are less than 11% of the main peak.

In some embodiments, the oxygenated hemoglobin composition is obtained from heat treated deoxygenated hemoglobin; wherein the heat treatment is performed at least for about 4 hours.

In some embodiments, the present invention provides a hemoglobin composition comprising:
a) oxygenated hemoglobin comprising main peak purity not less than 70%;
b) acidic variants are less than 12% of the main peak; and
c) basic variants are less than 11% of the main peak, wherein the oxygenated hemoglobin composition is obtained from heat treated deoxygenated hemoglobin; wherein the heat treatment is performed at least for 4 hours.

In some embodiments, the present invention provides a hemoglobin composition comprising:
a) oxygenated hemoglobin comprising main peak purity not less than 70%;
b) acidic variants are less than 12% of the main peak; and
c) basic variants are less than 9% of the main peak.

In some embodiments, the oxygenated hemoglobin composition is obtained from heat treated deoxygenated hemoglobin; wherein the heat treatment is performed at least for 4 hours.

In some embodiments, the present invention provides a hemoglobin composition comprising:
a) oxygenated hemoglobin comprising main peak purity not less than 70%;
b) acidic variants are less than 12% of the main peak; and
c) basic variants are less than 9% of the main peak,
   wherein the oxygenated hemoglobin composition is obtained from heat treated deoxygenated hemoglobin; wherein the heat treatment is performed at least for 4 hours.

In some embodiments, the oxygenated hemoglobin composition is obtained from heat treated deoxygenated hemoglobin; wherein the heat treatment is performed for suitable hours selected from 4 hours, 5 hours, 6 hours, 7 hours 8 hours, 9 hours, 10 hours, 11 hours, 12 hours, 13 hours, 14 hours. In some embodiments, the oxygenated hemoglobin composition is obtained from heat treated deoxygenated hemoglobin, wherein the heat treatment is performed for about 4 hours, about 5 hours, about 6 hours, about 7 hours, about 8 hours, about 9 hours, about 10 hours, about 11 hours, about 12 hours, about 13 hours, or about 14 hours.

In some embodiments, the present invention provides a process for preparation of stable hemoglobin composition comprising:
a) washing a fresh whole blood collected from animal sources to produce washed RBCs;
b) extracting a hemoglobin from the RBC's;
c) performing a filtration of the extracted hemoglobin;
d) performing ultrafiltration and concentration;
e) performing deoxygenation of ultrafiltered and concentrated hemoglobin;
f) performing heat inactivation of deoxygenated hemoglobin for reduction of virus and/or prion by heat treatment at suitable temperature;
g) performing reoxygenation of heat-treated deoxygenated hemoglobin to produce oxygenated hemoglobin composition;
h) optionally performing PEGylation of the oxygenated hemoglobin composition; and
i) optionally performing carboxylation process to produce carboxylated PEGylated hemoglobin composition,

In some embodiments, the viral inactivation of deoxygenated hemoglobin is performed by heat treatment. In some embodiments, the L-cysteine concentration in deoxygenated hemoglobin during step f) is maintained for more than 5 mM. In some embodiments, the oxygenated hemoglobin composition comprises main peak purity of more than 70% and one or more impurity selected from metHb, acidic variants, basic variants, virus particles and prions. In some embodiments, the oxygenated hemoglobin composition comprising not more than 2% metHb of total oxygenated hemoglobin.

In some embodiments, the present invention provides a process for preparation of stable hemoglobin composition comprising:
a) washing a fresh whole blood collected from animal sources to produce washed RBCs;
b) extracting a hemoglobin from the RBC's;
c) performing a filtration of the extracted hemoglobin;
d) performing ultrafiltration and concentration;
e) performing deoxygenation of ultrafiltered and concentrated hemoglobin;
f) performing heat inactivation of deoxygenated hemoglobin for reduction of virus and/or prion by heat treatment at suitable temperature;
g) performing reoxygenation of heat-treated deoxygenated hemoglobin to produce oxygenated hemoglobin composition;
h) optionally performing PEGylation of the oxygenated hemoglobin composition; and
i) optionally performing carboxylation process to produce carboxylated PEGylated hemoglobin composition,
   wherein the viral inactivation of deoxygenated hemoglobin is performed by heat treatment and maintains L-cysteine concentration in deoxygenated hemoglobin during step f) for more than 5 mM; wherein the oxygenated hemoglobin composition comprises main peak purity of more than 70% and one or more impurity selected from metHb, acidic variants, basic variants, virus particles and prions; wherein the oxygenated hemoglobin composition comprising not more than 2% metHb of total oxygenated hemoglobin.

In some embodiments, the present invention provides a process for reducing the viral and/or prion load in the oxygenated hemoglobin composition obtained from mammalian sources comprising:
a) performing heat treatment of deoxygenated hemoglobin at about 60° C. at least for 4 hours;
b) maintains L-cysteine concentration more than 5.0 mM during the heat treatment;
c) performing the reoxygenation of the heat-treated deoxygenated hemoglobin to form a oxygenated hemoglobin composition;
d) optionally performing PEGylation of the oxygenated hemoglobin composition; and
e) optionally performing carboxylation process to produce carboxylated PEGylated hemoglobin composition.

In some embodiments, the present invention provides a process for the preparation of stable hemoglobin composition comprising:
a) washing a fresh whole blood collected from animal sources to produce washed RBCs;
b) extracting a hemoglobin from the RBC's;
c) performing a filtration of the extracted hemoglobin;
d) performing ultrafiltration and concentration;
e) performing deoxygenation of ultrafiltered and concentrated hemoglobin;
f) performing viral inactivation of deoxygenated hemoglobin for reduction of virus and/or prion by heat treatment at suitable temperature with suitable concentration of antioxidant more than 5 mM;
g) performing reoxygenation of heat-treated deoxygenated hemoglobin composition to produce oxygenated hemoglobin composition;
h) optionally performing PEGylation of the heat treated oxygenated hemoglobin composition; and
i) optionally performing carboxylation process to produce carboxylated PEGylated hemoglobin composition.

In some embodiments, the oxygenated hemoglobin composition has lower charge variants in comparison to charge variants measured in oxygenated hemoglobin composition prepared by process with antioxidant at or less than 5 mM.

In some embodiments, the present invention provides a process for the preparation of stable hemoglobin composition comprising:
a) washing a fresh whole blood collected from animal sources to produce washed RBCs;
b) extracting a hemoglobin from the RBC's;
c) performing a filtration of the extracted hemoglobin;
d) performing ultrafiltration and concentration;
e) performing deoxygenation of ultrafiltered and concentrated hemoglobin;
f) performing viral inactivation of deoxygenated hemoglobin for reduction of virus and/or prion by heat treatment at suitable temperature with suitable concentration of antioxidant more than 5 mM;
g) performing reoxygenation of heat-treated deoxygenated hemoglobin composition to produce oxygenated hemoglobin composition;
h) optionally performing PEGylation of the heat treated oxygenated hemoglobin composition; and
i) optionally performing carboxylation process to produce carboxylated PEGylated hemoglobin composition,
   wherein the oxygenated hemoglobin composition has lower charge variants in comparison to charge variants measured in oxygenated hemoglobin composition prepared by process with antioxidant at or less than 5 mM.

In some embodiments, the antioxidant is L-cysteine.

In some embodiments, the present invention provides a process for controlling and/or reducing formation of metHb during the manufacturing of oxygenated hemoglobin composition comprising:
a) performing deoxygenation of hemoglobin to form a deoxygenated hemoglobin;
b) heat treatment of the deoxygenated hemoglobin with about 5.5 mM to about 15 mM of L-cysteine;
c) reoxygenation of the deoxygenated hemoglobin to form an oxygenated hemoglobin composition;
d) measuring the metHb concentration in the oxygenated hemoglobin composition;
   wherein the oxygenated hemoglobin composition has less than 2% of metHb formation analyzed by co-oximetry;
e) optionally performing PEGylation of the oxygenated hemoglobin composition; and
f) optionally performing carboxylation process to produce a carboxylated PEGylated hemoglobin composition.

In some embodiments, the pegylated hemoglobin composition of step (e) or Pegylated carboxylated hemoglobin composition of step (f) maintains the metHb below 4%, preferably below 3%.

In some embodiments, the present invention provides a process for controlling and/or reducing formation of metHb during the manufacturing of oxygenated hemoglobin composition comprising:
a) performing deoxygenation of hemoglobin to form a deoxygenated hemoglobin;
b) heat treatment of the deoxygenated hemoglobin with about 5.5 mM to about 15 mM of L-cysteine;
c) reoxygenation of the deoxygenated hemoglobin to form an oxygenated hemoglobin composition;
d) measuring the metHb concentration in the oxygenated hemoglobin composition;
   wherein the oxygenated hemoglobin composition has less than 2% of metHb formation analyzed by co-oximetry;
g) optionally performing PEGylation of the oxygenated hemoglobin composition; and
h) optionally performing carboxylation process to produce a carboxylated PEGylated hemoglobin composition,
   wherein the pegylated hemoglobin composition of step (e) or Pegylated carboxylated hemoglobin composition of step (f) maintains the metHb below 4%, preferably below 3%.

In some embodiments, the present invention provides a process for the preparation of stable hemoglobin composition comprising:
a) washing a fresh whole blood collected from animal sources to produce washed RBCs;
b) extracting a hemoglobin from the RBC's;
c) performing a filtration of the extracted hemoglobin;
d) performing ultrafiltration and concentration;
e) performing deoxygenation of ultrafiltered and concentrated hemoglobin;
f) performing viral inactivation of deoxygenated hemoglobin for reduction of virus and/or prion by heat treatment at suitable temperature;
g) performing reoxygenation of heat-treated deoxygenated hemoglobin to produce oxygenated hemoglobin composition;
h) optionally performing PEGylation of the heat treated deoxygenated hemoglobin; and
i) optionally performing carboxylation process to produce carboxylated PEGylated hemoglobin composition.

In some embodiments, the oxygenated hemoglobin composition has lower charge variants in comparison to charge variants measured in oxygenated hemoglobin composition prepared by process with antioxidant at or less than 5 mM.

In some embodiments, the present invention provides a process for the preparation of stable hemoglobin composition comprising:
a) washing a fresh whole blood collected from animal sources to produce washed RBCs;
b) extracting a hemoglobin from the RBC's;
c) performing a filtration of the extracted hemoglobin;
d) performing ultrafiltration and concentration;
e) performing deoxygenation of ultrafiltered and concentrated hemoglobin;
f) performing viral inactivation of deoxygenated hemoglobin for reduction of virus and/or prion by heat treatment at suitable temperature;
g) performing reoxygenation of heat-treated deoxygenated hemoglobin to produce oxygenated hemoglobin composition;
h) optionally performing PEGylation of the heat treated deoxygenated hemoglobin;
i) optionally performing carboxylation process to produce carboxylated PEGylated hemoglobin composition;
   wherein the oxygenated hemoglobin composition has lower charge variants in comparison to charge variants measured in oxygenated hemoglobin composition prepared by process with antioxidant at or less than 5 mM.

In some embodiments, the present invention provides a hemoglobin composition comprising:
a) oxygenated hemoglobin; and
b) metHb less than 9% of total oxygenated hemoglobin.

In some embodiments, the oxygenated hemoglobin is subjected to heat treatment for about 12 hours. In some embodiments, the oxygenated hemoglobin is subjected to heat treatment for about 11 hours. In some embodiments, the oxygenated hemoglobin is subjected to heat treatment for about 10 hours. In some embodiments, the oxygenated hemoglobin is subjected to heat treatment for about 9 hours. In some embodiments, the oxygenated hemoglobin is subjected to heat treatment for about 8 hours. In some embodiments, the oxygenated hemoglobin is subjected to heat treatment for about 7 hours. In some embodiments, the oxygenated hemoglobin is subjected to heat treatment for about 6 hours

In some embodiments, a hemoglobin composition comprising:
c) oxygenated hemoglobin; and
d) metHb less than 9% of total oxygenated hemoglobin,
   wherein the heat treatment is performed for about 10 hours.

In some embodiments, the present invention provides a hemoglobin composition comprising:
a) oxygenated hemoglobin; and
b) metHb less than 4% of total oxygenated hemoglobin;
   wherein the heat treatment is performed for about 8 hours.

In some embodiments, the composition comprising an oxygenated hemoglobin that comprises main peak hemoglobin purity of more than 70%, less than 2% metHb, less than 25% charge variants, wherein the charge variants are acidic variant or basic variant of that main peak.

In some embodiments, the main peak purity of oxygenated hemoglobin is analyzed by cIEF (capillary Iso Electric Focusing) and metHb analyzed by co-oximetry.

In some embodiments, the acidic and basic variants of oxygenated hemoglobin composition is analyzed by cIEF (capillary Iso Electric Focusing).

In some embodiments, the oxygenated hemoglobin composition comprises total percentage of metHb less than 2%. In some embodiments, the oxygenated hemoglobin composition comprises total percentage of metHb selected from about 0.5% to about 1.8% of total oxygenated hemoglobin.

In some embodiments, the stable hemoglobin composition is manufactured at large scale.

In some embodiments, the large scale is more than 20 L. In certain embodiment, the large scale is more than 30 L, 50 L, 100 L, 200 L, 500 L, 1000 L, 5000 L.

In some embodiments, the purity of oxygenated hemoglobin composition is more than about 70%, about 71%, about 72%, about 73%, about 74%, about 75%, about 76%, about 77%, about 78%, about 79%, about 80%, about 81%, about 82%, about 83%, about 84%, and about 85% or more.

In some embodiments, the acidic variants are less than about 20%. In some embodiments, the acidic variants are less than about 12%, less than about 11%, less than about 10%, less than about 9%, less than about 8%, less than about 7% or less. In some embodiments, the basic variants are less than about 11%, less than about 10%, less than about 9%, less than about 8%, less than about 7% or less.

In some embodiments, the viral reduction of heat-treated deoxygenated hemoglobin has been reduced by a factor of more than 1 log₁₀, more than 2 log₁₀ or more in comparison with the process performed without the heat treatment.

In some embodiments, the prion reduction of heat-treated deoxygenated hemoglobin has been reduced by a factor of more than 1 log₁₀, more than 2 log₁₀, more than 3 log₁₀, more than 4 log₁₀ or more in comparison with the process performed without the heat treatment.

In some embodiments, the L-cysteine suitable concentration is more than 5 mM. In some embodiments, the L-cysteine suitable concentration is selected from about 5.5 mM to about 15 mM.

In some embodiments, the heat treatment process is carried out at suitable temperature selected from about 55° C. to about 75° C.

In some embodiments, the heat treatment process is carried out at least more than 4 hours.

In some embodiments, the heat treatment process is carried out at a suitable time period selected from about 4 hours to about 15 hours.

In some embodiments, the present invention provides a method of treating a condition that can be ameliorated by oxygenating the red blood cells of a patient in need of such treatment, by administering to patient a therapeutically effective amount of stable hemoglobin composition comprising:
a) oxygenated hemoglobin comprising main peak purity not less than 70%; and
b) one or more impurity selected from metHb, acidic variants, basic variants, virus particles and/or prions,

In some embodiments, the oxygenated hemoglobin comprising not more than 2% metHb of total oxygenated hemoglobin.

In some embodiments, the present invention provides a method of treating a condition that can be ameliorated by oxygenating the red blood cells of a patient in need of such treatment, by administering to patient a therapeutically effective amount of stable hemoglobin composition comprising:
a) oxygenated hemoglobin comprising main peak purity not less than 70%; and
b) one or more impurity selected from metHb, acidic variants, basic variants, virus particles and/or prions,
   wherein the oxygenated hemoglobin comprising not more than 2% metHb of total oxygenated hemoglobin.

In some embodiments, the present invention provides a method of treating a condition that can be ameliorated by oxygenating the red blood cells of a patient in need of such treatment, by administering to patient a therapeutically effective amount of stable hemoglobin composition comprising:
a) oxygenated hemoglobin comprising not more than 2% metHb;
b) oxygenated hemoglobin comprising main peak purity not less than 70%;
c) oxygenated hemoglobin comprising less than 12% acidic variants of the main peak;
d) oxygenated hemoglobin comprising less than 11% basic variants of the main peak; and
e) oxygenated hemoglobin comprising substantially free of virus particles and/or prion.

In some embodiments, the condition of patient is selected from the group comprising acute respiratory distress syndrome, bronchiectasis, bronchopulmonary dysplasia, chronic obstructive pulmonary disease, cystic fibrosis, emphysema, lymphangiomatosis, primary ciliary dyskinesia, cancer, tumour, cancers of the lung, pulmonary hypertension, pulmonary fibrosis, pulmonary vascular disease, pulmonary sarcoidosis, pneumonia and bronchitis, infectious diseases that affect the lung's ability to transport.

In some embodiments, the condition is an anemia selected from the group consisting of blood loss anemias, anemias caused by faulty red blood cell production, anemias caused by red blood cell destruction, and a combination thereof; wherein the cancers are selected from solid tumors, soft tissue carcinoma, lung cancer, bone cancer, metastatic cancer, adrenal cancer, anal cancer, appendix cancer, bile duct cancer, bladder cancer, bone cancer, brain cancer, breast cancer.

In some embodiments, the present invention provides a method of administration of a stable hemoglobin composition comprising:
a) oxygenated hemoglobin comprising main peak purity not less than 70%; and
b) one or more impurity selected from metHb, acidic variants, basic variants, virus particles and/or prions.

In some embodiments, the oxygenated hemoglobin comprising not more than 2% metHb of total oxygenated hemoglobin.

In some embodiments, the present invention provides a method of administration of a stable hemoglobin composition comprising:
c) oxygenated hemoglobin comprising main peak purity not less than 70%; and
d) one or more impurity selected from metHb, acidic variants, basic variants, virus particles and/or prions,
   wherein the oxygenated hemoglobin comprising not more than 2% metHb of total oxygenated hemoglobin.

In some embodiments, the present invention provides a method of administration of a stable hemoglobin composition comprising:
a) oxygenated hemoglobin comprising not more than 2% metHb;
b) oxygenated hemoglobin comprising main peak purity not less than 70%;
c) oxygenated hemoglobin comprising less than 12% acidic variants of the main peak;
d) oxygenated hemoglobin comprising less than 11% basic variants of the main peak;
e) oxygenated hemoglobin comprising substantially free of virus particles and/or prion;
   wherein, metHb is analyzed by co-oximetry and oxygenated hemoglobin main peak purity is analyzed by cIEF (capillary Iso Electric Focusing).

In some embodiments, the dosing frequency is once a daily or twice a daily.

In some embodiments, the present invention provides a stable composition of hemoglobin comprising:
a) reoxygenated hemoglobin containing not more than 2% MetHb; and
b) reoxygenated hemoglobin main peak purity not less than 60%;
   wherein, MetHb is analyzed by co-oximetry and reoxygenated hemoglobin main peak purity is analyzed by cIEF (capillary Iso Electric Focusing).

In some embodiments, the present invention provides a heat treatment process for eliminating or reducing potential endogenous adventitious agents in hemoglobin composition by using L-cysteine in suitable concentration.

In some embodiments, the L-cysteine suitable concentration is more than 5 mM. In some embodiments, the L-cysteine suitable concentration is selected from about 5.5 mM to about 15 mM.

In some embodiments, the invention provides a stable hemoglobin composition comprising;
a. performing the heat treatment of deoxyhemoglobin composition at suitable temperature for suitable period of time;
b. maintains suitable concentration of antioxidant more than 5 mM;
c. reoxygenation of the hemoglobin composition; and
d. measuring the MetHb concentration in reoxygenated hemoglobin composition;
   wherein the reoxygenated hemoglobin composition has lower concentration of the MetHb in comparison to MetHb concentration measured in reoxygenated hemoglobin composition treated with antioxidant at or less than 5 mM.

In some embodiments, the invention performs reoxygenation of hemoglobin composition post heat treatment wherein the hemoglobin composition maintains concentration of MetHb is below 2% analyzed by co-oximetry.

In some embodiments, the heat treatment process is carried out at suitable temperature selected from above 59° C. to about 65° C.

In some embodiments, the heat treatment process is carried out at a suitable time period selected from about 5 hours to about 10 hours.

In some embodiments, the invention can be applied to eliminate or reduce all virus forms, particularly DNA and RNA viruses, enveloped and non-enveloped viruses, furthermore to virions and prions or other similar biological systems and also bacteria and fungi. The method is preferably used to reduce the Bovine viral diarrheal virus (BVDV) virus, Retrovirus, Parvovirus contamination, Bovine spongiform encephalopathy (BSE), and Transmissible spongiform encephalopathies (TSEs) contamination from the hemoglobin solution.

In some embodiments, the invention provides a stable hemoglobin composition comprising:
a) reoxygenated Hb purity more than 60%;
b) reoxygenated Hb substantially free of virus particles; and
c) reoxygenated Hb substantially free from prions.

In some embodiments, viral load of heat-treated hemoglobin solution has been reduced by a factor of more than 1 log₁₀, more than 2 log₁₀, more than 3 log₁₀, more than 4 log₁₀ or more in comparison with before the heat treatment.

In some embodiments, the invention provides a process for the preparation of stable hemoglobin composition comprising:
a. performing the heat treatment of deoxyhemoglobin composition at suitable temperature for suitable period of time;
b. maintains suitable concentration of antioxidant more than 5 mM;
c. reoxygenation of hemoglobin composition; and
d. measuring the MetHb concentration in reoxygenated hemoglobin composition,
   wherein the reoxygenated hemoglobin composition has lower concentration of the MetHb in comparison to MetHb concentration measured in reoxygenated hemoglobin composition prepared by process with antioxidant at or less than 5 mM.

In certain embodiment the invention provides a process for the preparation of stable hemoglobin composition comprising:
a. performing the heat treatment of deoxyhemoglobin composition at suitable temperature for suitable period of time;
b. maintains suitable concentration of antioxidant more than 5 mM;
c. reoxygenation of the hemoglobin composition;
d. measuring the charge variants in the reoxygenated hemoglobin composition,
   wherein the reoxygenated hemoglobin composition has lower charge variants in comparison to charge variants measured in reoxygenated hemoglobin composition prepared by process with antioxidant at or less than 5 mM.

### BRIEF DESCRIPTION OF THE DRAWINGS

FIG. 1 depicts the effects of L-Cysteine concentration vs % metHb formation during reoxygenation/oxygenation.

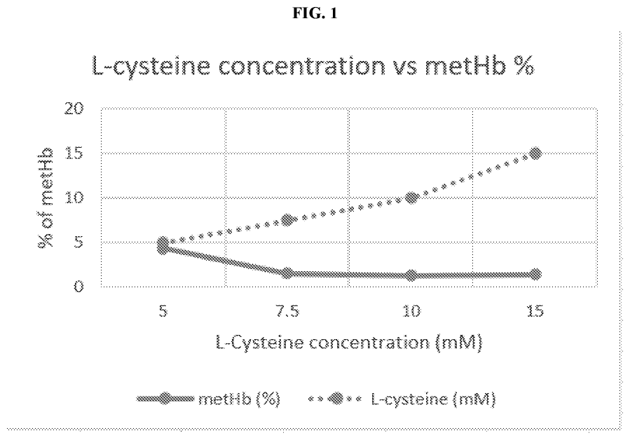

FIG. 2A depicts the capillary Iso Electric Focusing (cIEF-Maurice) profiles of typical hemoglobin pre-heat treatment in presence of 5 mM L-cysteine.

FIG. 2B depicts the capillary Iso Electric Focusing (cIEF-Maurice) profiles after treatment at 60° C. for 10 hours in presence of 5 mM L-cysteine showing elevation in acidic and basic charge variants.

FIG. 2C depicts the capillary Iso Electric Focusing (cIEF-Maurice) profiles after treatment at 60° C. for 10 hours in presence of 15 mM L-cysteine showing no elevation in charge variants.

FIG. 3 depicts the effects of varying L-cysteine concentration with respect to oxygenated hemoglobin purity/main peak purity during heat treatment analyzed by capillary Iso Electric Focusing (CIEF).

### DETAILED DESCRIPTION OF THE INVENTION

The present invention provides an effective control of metHb formation during manufacturing or purification process of oxygenated hemoglobin (e.g., reoxygenated hemoglobin). In addition, the present invention also provides an effective control of charge variants and virus and/or prions during manufacturing or purification process of reoxygenated/oxygenated hemoglobin.

According to the present invention, the metHb percentage of reoxygenated/oxygenated hemoglobin is less than about 2% (e.g., less than about 2%, less than about 1.9%, less than about 1.8%, less than about 1.7%, less than about 1.6%, less than about 1.5%, less than about 1.4%, less than about 1.3%, less than about 1.2%, less than about 1.1%, less than about 1.0%, less than about 0.95%, less than about 0.9%, less than about 0.85%, less than about 0.8%, less than about 0.75%, less than about 0.7%, less than about 0.65%, less than about 0.6%, less than about 0.55%, less than about 0.5%, less than about 0.45%, less than about 0.4%, less than about 0.35%, less than about 0.3%, less than about 0.25%, less than about 0.2%, less than about 0.15%, less than about 0.1%, or less than about 0.05%).

The present invention addresses the need for an effective control of metHb formation in reoxygenated/oxygenated hemoglobin composition by using a specific amount or concentration of an antioxidant (e.g., cysteine, e.g., L-cysteine).

The present invention also provides the stability of hemoglobin in high temperature during heat treatment.

The term “comprises” or “comprising” is used in the present description, it does not exclude other elements or steps. For the purpose of the present invention, the term “consisting of” is considered to be an optional embodiment of the term “comprising of”. If hereinafter a group is defined to comprise at least a certain number of embodiments, this is also to be understood to disclose a group which optionally consists only of these embodiments.

As used throughout the specification and in the appended claims, the singular forms “a,” “an,” and “the” include the plural reference unless the context clearly dictates otherwise.

The term “about”, as used herein, is intended to refer to ranges of approximately 10-20% greater than or less than the referenced value. In certain circumstances, one skill in the art will recognize that, due to the nature of the referenced value, the term “about” can mean more or less than a 10-20% deviation from that value.

The term “substantially free” used herein refers to hemoglobin composition that is free of one or more impurities selected from metHb, virion, prion or such adventitious agents, charge variants and has purity above about 60% (e.g., above about 65%, above about 70%, above about 75%, above about 80%, above about 85%, above about 90%, or above about 95%). In some embodiments, the term “substantially free of virus and/or prions” refers to the purified hemoglobin composition wherein the purified hemoglobin composition is free from adventitious agents or viruses or prions more than about 95%, more than about 96%, more than about 97%, more than about 98%, or more than about 99%. In some embodiments, the term “substantially free of virus and/or prions” refers to reduction of virus and/or prions more than 1 log₁₀, more than 2 log₁₀, more than 3 log₁₀, more than 4 log₁₀ or more post heat treatment.

The term “hemoglobin” or “Hb” used herein are interchangeable refers generally to the protein contained within red blood cells that transports oxygen. Hemoglobin of use in the present invention is derived from substantially any mammalian source. Exemplary sources of hemoglobin include common livestock animals, e.g., cows, pigs, sheep and the like. The invention is not limited by the source of the hemoglobin. In various embodiments, the hemoglobin is bovine hemoglobin.

The term “hemoglobin composition” refers to composition of hemoglobin having desired amount of metHb wherein the hemoglobin is reoxygenated hemoglobin. In some embodiments, hemoglobin composition is considered stable when the hemoglobin composition comprising not more than 2% metHb. In some embodiments, hemoglobin composition comprising not more than 2% metHb and main purity peak of hemoglobin not less than 70%. In some embodiments, hemoglobin composition comprising charge variants less than 25% of the main peak of the reoxygenated hemoglobin. The charge variants comprise acidic and basic variants having less than 12% of the main peak. In some embodiments, the hemoglobin composition comprising substantially free of virus particles and prions. In certain embodiment, the hemoglobin composition has pH 7.4 to pH 8.2. In some embodiments, the PEGylated and/or carboxylated hemoglobin composition pH is 7.4 to pH 8.0.

The term “tHb” used herein refers to the total hemoglobin content during the process of manufacturing purified hemoglobin.

The term “stable hemoglobin” composition refers to the hemoglobin composition provides stability of the hemoglobin composition from batch to batch and thereby helps to achieve batch to batch consistency. To provide further clarity, stable hemoglobin composition maintains main purity peak of hemoglobin/reoxygenated hemoglobin at desired level at more than 70% analyzed by cIEF and metHb less than 2% analyzed by co-oximetry which is also helpful in long term storage of PEG-hemoglobin or PEG-Hb-CO composition. To provide clarity, stable hemoglobin referred herein relates to oxygenated hemoglobin which is not PEGylated. PEGylated hemoglobin or carboxylated hemoglobin is produced by conjugating PEG molecule to oxygenated hemoglobin. The PEGylated hemoglobin or carboxylated hemoglobin (PEG-Hb-CO) maintains less than 5% metHb during storage when metHb is controlled less than 2% during manufacturing of oxygenated hemoglobin. In certain embodiments, The PEGylated hemoglobin or carboxylated hemoglobin (PEG-Hb-CO) maintains less than 4% metHb during storage when metHb is controlled less than 2% during manufacturing of oxygenated hemoglobin. In some embodiments, The PEGylated hemoglobin or carboxylated hemoglobin (PEG-Hb-CO) maintains less than 3% metHb during storage when metHb is controlled less than 2% during manufacturing of oxygenated hemoglobin.

In some embodiments, oxygenated hemoglobin composition maintains metHb below 2% during storage at 2-8° C. for more than 1 day, 3 days, 7 days, 1 months, and 3 months. In some embodiments, oxygenated hemoglobin composition maintains metHb below 2% during storage at less than −70° C. for more than 1 month, 3 months, 6 months, 9 months, and 12 months. In some embodiments, the p50 value of reoxygenated/oxygenated hemoglobin is 7-16 mmHg analyzed by Hemox Analyzer.

In some embodiments, the PEGylated hemoglobin or carboxylated hemoglobin (PEG-Hb-CO) composition maintains metHb below 5%, preferably below 4% during storage at 2-8° C. for more than 1 month, 3 months, 6 months, 9 months, 12 months, 15 months, 18 months, 21 months, 24 months, 27 months, 30 months, 33 months, 36 months, 39 months, 42 months, 45 months, and 48 months. In some embodiments, the p50 value of reoxygenated/oxygenated hemoglobin is 7-16 mmHg analyzed by Hemox Analyzer.

In some embodiments, the PEGylated hemoglobin or carboxylated hemoglobin (PEG-Hb-CO) composition maintains metHIb below 5%, preferably below 4% during storage at 25° C. for more than 1 month, 3 months, 6 months, 9 months, 12 months, 15 months, 18 months, 21 months, 24 months, 27 months, 30 months, 33 months, 36 months, 39 months, 42 months, 45 months, and 48 months. In some embodiments, the p50 value of reoxygenated/oxygenated hemoglobin is 7-16 mmHg analyzed by Hemox Analyzer.

In some embodiments, the PEGylated hemoglobin or carboxylated hemoglobin (PEG-Hb-CO) composition maintains metHb below 5%, preferably below 4% during storage at 40° C. for more than 1 month, 3 months, 6 months, 9 months, and 12 months. In some embodiments, the p50 value of reoxygenated/oxygenated hemoglobin is 7-16 mmHg analyzed by Hemox Analyzer.

In some embodiments, the PEGylated hemoglobin or carboxylated hemoglobin (PEG-Hb-CO) composition maintains metHb below 3% during storage at 2-8° C. for more than 1 month, 3 months, 6 months, 9 months, 12 months, 15 months, 18 months, 21 months, 24 months, 27 months, 30 months, 33 months, 36 months, 39 months, 42 months, 45 months, and 48 months. In some embodiments, the p50 value of reoxygenated/oxygenated hemoglobin is 7-16 mmHg analyzed by Hemox Analyzer.

The term “reoxygenation” is referred to a process of attaching oxygen (O₂) molecule to the deoxygenated hemoglobin by methods generally known in the art e.g., diafiltration etc.

The term “reoxygenated Hb” or “reoxygenated hemoglobin” or “oxygenated hemoglobin” or “oxygenated Hb” used herein are interchangeable and refers to the oxygenated hemoglobin molecule prepared post viral inactivation or post heat treatment by attaching with oxygen (O₂) that was deoxygenated prior viral inactivation or prior heat treatment. In some embodiments, the reoxygenation is performed methods generally known in the art.

The term “heat treated oxygenated hemoglobin” or “reoxygenated hemoglobin” or “oxygenated hemoglobin” are interchangeable and refers to the hemoglobin of post reoxygenation/oxygenation step.

The term “post reoxygenation” refers to the hemoglobin composition after performing diafiltration or after performing reoxygenation step. The reoxygenation of hemoglobin is performed with a buffer solution by diafiltration generally known in the art.

Ther term “PEG-hemoglobin” or “PEG-Hb” are interchangeable and refers to reoxygenated/oxygenated hemoglobin which is attached with the PEG molecule to improve the half-life of oxygenated hemoglobin. PEGylation process is the conjugation of eight to ten chains of 5 kD activated polyethylene glycol (SC-PEG-5K) molecules to the purified reoxygenated/oxygenated hemoglobin. The PEG-Hb is capable to deliver oxygen to tissues. Alternatively, PEG-Hb can be bound to carbon monoxide (CO) instead of oxygen binding refers to PEG-Hb-CO. In some embodiments, the p50 value of PEG-hemoglobin or PEG-Hb-CO is 7-16 mmHg analyzed by Hemox Analyzer. In some embodiments, PEG-hemoglobin or PEG-Hb-CO composition comprises metHb less than 2%. In some embodiments, PEG-hemoglobin or PEG-Hb-CO composition comprises metHb about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, and about 1.8% of total PEG-Hb. It is observed that metHb is maintained below 3% during the long-term storage of PEG-Hb at 2-8° C. when the metHb is controlled below 2% during manufacturing of PEG-Hb. In certain embodiment, PEG-hemoglobin or PEG-Hb-CO composition maintains metHb below 2% during storage at 2-8° C. more than 1 month, 3 months, 6 months, 9 months, 12 months, 15 months, 18 months, 21 months, 24 months, 27 months, 30 months, 33 months, 36 months, 39 months, 42 months, 45 months, and 48 months.

The term “deoxygenated hemoglobin” or “deoxygenated Hb” used herein are interchangeable and refers to hemoglobin in which the Fe (II) atom is bound to a species other than oxygen (e.g., carbon monoxide) or is not bound to oxygen or any other species. The deoxygenation of hemoglobin is performed by passage through a counter-current membrane contactor (Liqui-Cel®). The deoxygenation of hemoglobin is performed prior to heat treatment.

The term “carboxylated” refers to gas bound hemoglobin. In some embodiments, the gas bound hemoglobin has CO attached to hemoglobin.

The term “antioxidant” used herein refers to a molecule (e.g., an amino acid) capable to inhibit or reverse the oxidation reaction. In the present invention, antioxidant is used to prevent or reduce the formation of impurities associated with the hemoglobin wherein the one or more impurity is selected from metHb, acidic variants, and basic variants. In certain embodiments, antioxidants are helpful during heat treatment of hemoglobin/deoxygenated hemoglobin performed to reduce virus and/or prions. In some embodiments, antioxidants are selected from N-acetyl cysteine, glutathione, ascorbic acid, preferably L-cysteine. In some embodiments, the antioxidant is used more than 5 mM during heat treatment.

The term “cIEF” or “capillary Iso Electric Focusing” used herein are interchangeable refers to the analytical method that determines the purity/impurity level in the hemoglobin solution. Capillary isoelectric focusing (cIEF) is a high-resolution analytical technique that allows the separation of protein/peptide mixtures, protein glycoforms and other charge variants, based on their isoelectric point (pl). The method can be performed by a skilled person.

The term “reduced” used herein refers to the decreasing of impurities formation ranges of approximately 60-80%.

The term “impurity” used herein refers to metHb—an inactive state of hemoglobin, charge variants and adventitious agents present due to source of material, viruses and/or prions etc.

The term “metHb” or “methemoglobin” refers to a dysfunctional form of hemoglobin (impurity) that is incapable of transporting oxygen.

metHb is determined by any art-recognized method of analysis. In the present invention, the metHb impurity is determined by co-oximetry.

The term “co-oximetry” used herein refers to the measurement of various forms of hemoglobin by dedicated multiwavelength spectrophotometry. It is a measure of the potential oxygen-carrying capacity of the blood. CO-oximeters are multiwavelength spectrophotometers that measure the optical absorbance of blood at different wavelengths and automatically calculate the fractional concentration of the four major Hb species (oxy-, deoxy-, carboxy-, and methemoglobin) from a total Hb concentration.

The term “main peak purity” or “purity” are interchangeable and refers to the total percentage hemoglobin without metHb and other impurities comprising charge variants, adventitious agents that includes virus and/or prions.

Purity is determined by any art-recognized method of analysis (e.g., Capillary Iso Electric Focusing, band intensity on a silver-stained gel, polyacrylamide gel electrophoresis, HPLC, or a similar means).

The term “charge variants” includes product related impurity. In certain embodiment, the charge variants include acidic variants &/or basic variants.

The term “acidic variants” or “acidic species” and “AV” used herein refer to the variants of a protein, which are characterized by an overall acidic charge.

The term used “basic variants” or “basic species” refers to variants can result from the presence of C-terminal lysine additional positive charges or removal of negative charges.

The acidic and basic charge variants are analyzed by cIEF (capillary Iso Electric Focusing) method generally known in the art.

The term “heat treatment” refers to a method or process that is carried out at a particular temperature suitable for inactivation of viruses and/or prions during the preparation of oxygenated hemoglobin, preferably carried out at a temperature from about 55° C. to about 75° C., particularly preferably at 60±1° C. It is observed that heat treatment causes the formation of impurities in oxygenated hemoglobin. In certain embodiment, the heat treatment is carried out with deoxygenated hemoglobin. The term “high temperature” used herein refers to the temperature from about 55° C. to 75° C.

The term “large scale” refers to industrial scale more than at scale of 10 L. In embodiment, the large scale is selected from 20 L to 1000 L. In certain embodiment, the large scale is more than 30 L, 50 L, 100 L, 200 L, 500 L, 1000 L, 5000 L. In one aspect of such embodiment the invention provides batch to batch consistency at large scale. The invention maintains impurities in acceptable limits required by regulatory body.

The meaning of other terminology used herein should be easily understood by someone of reasonable skill in the art.

In some embodiments, the present invention provides a hemoglobin composition comprising:
a) oxygenated hemoglobin; and
b) metHb less than 2% of total oxygenated hemoglobin.

In such embodiment, oxygenated hemoglobin comprises main peak purity not less than 70%.

In some embodiments, the present invention provides a hemoglobin composition comprising:
a) oxygenated hemoglobin comprising main peak purity not less than 70%;
b) metHb less than 2% of total oxygenated hemoglobin.

In some embodiments, the present invention provides a composition of hemoglobin comprising:
a) oxygenated hemoglobin comprising main peak purity not less than 70%;
b) one or more impurity selected from metHb, charge variants selected from acidic variants, basic variants and optionally virus particles and/or prions;
   wherein the oxygenated hemoglobin comprising not more than 2% metHb of total oxygenated hemoglobin.

In some embodiments, the present invention provides a composition of hemoglobin comprising:
a) oxygenated hemoglobin comprising main peak purity not less than 70% and one or more impurity comprising;
b) oxygenated hemoglobin comprising not more than 2% metHb;
c) oxygenated hemoglobin comprising less than 12% acidic variants of the main peak;
d) oxygenated hemoglobin comprising less than 11% basic variants of the main peak;
e) oxygenated hemoglobin comprising substantially free of virus particles; and
f) oxygenated hemoglobin comprising substantially free of prions;
   wherein, metHb is analyzed by co-oximetry and oxygenated hemoglobin main peak purity is analyzed by cIEF (capillary Iso Electric Focusing).

In some embodiments, the present invention provides a hemoglobin composition comprising:
a) oxygenated hemoglobin comprising main peak purity not less than 70%;
b) acidic variants are less than 12% of the main peak;
c) basic variants are less than 11% of the main peak;
   wherein the oxygenated hemoglobin composition is obtained from heat treated deoxygenated hemoglobin; wherein the heat treatment is performed at least for 4 hours.

In some embodiments, the oxygenated hemoglobin is obtained from heat treated deoxyhemoglobin; wherein the heat treatment is performed for suitable hours selected from 8 hours, 10 hours, 12 hours, and 14 hours.

In some embodiments, the present invention provides a hemoglobin composition comprising:
a) oxygenated hemoglobin comprising main peak purity not less than 70%;
b) acidic variants are less than 12% of the main peak;
c) basic variants are less than 9% of the main peak;
   wherein the oxygenated hemoglobin composition is obtained from heat treated deoxygenated hemoglobin; wherein the heat treatment is performed at least for 4 hours.

In some embodiments, the oxygenated hemoglobin is obtained from heat treated deoxyhemoglobin; wherein the heat treatment is performed for suitable hours selected from 4 hours, 5 hours, 6 hours, and 7 hours, 8 hours, 9 hours, 10 hours, 11 hours, 12 hours, 13 hours, and 14 hours.

In some embodiments, the present invention provides a process for preparation of stable hemoglobin composition comprising:
a) washing a fresh whole blood collected from animal sources to produce washed RBCs;
b) extracting a hemoglobin from the RBC's;
c) performing a filtration of the extracted hemoglobin;
d) performing ultrafiltration and concentration;
e) performing deoxygenation of ultrafiltered and concentrated hemoglobin;
f) performing heat inactivation of deoxygenated hemoglobin for reduction of virus and/or prion by heat treatment at suitable temperature;
g) performing reoxygenation of heat-treated deoxygenated hemoglobin to produce oxygenated hemoglobin composition;
h) optionally performing PEGylation of the oxygenated hemoglobin composition;
i) optionally performing carboxylation process to produce carboxylated PEGylated hemoglobin composition;
   wherein the viral inactivation of deoxygenated hemoglobin is performed by heat treatment and maintains L-cysteine concentration in deoxygenated hemoglobin during step f) for more than 5 mM; wherein the oxygenated hemoglobin composition comprises main peak purity of more than 70% and one or more impurity selected from metHb, acidic variants, basic variants, virus particles and prions; wherein the oxygenated hemoglobin composition comprising not more than 2% metHb of total oxygenated hemoglobin.

In some embodiments, the present invention provides a process for reducing the viral and/or prion load in the oxygenated hemoglobin composition obtained from mammalian sources comprising:
a) performing heat treatment of deoxygenated hemoglobin at about 60° C. at least for 4 hours;
b) maintains L-cysteine concentration more than 5.0 mM during the heat treatment;
c) performing the reoxygenation of the heat-treated deoxygenated hemoglobin to form a oxygenated hemoglobin composition;
d) optionally performing PEGylation of the oxygenated hemoglobin composition;
e) optionally performing carboxylation process to produce carboxylated PEGylated hemoglobin composition.

In some embodiments, the present invention provides a process for the preparation of stable hemoglobin composition comprising:
a) washing a fresh whole blood collected from animal sources to produce washed RBCs;
b) extracting a hemoglobin from the RBC's;
c) performing a filtration of the extracted hemoglobin;
d) performing ultrafiltration and concentration;
e) performing deoxygenation of ultrafiltered and concentrated hemoglobin;
f) performing viral inactivation of deoxygenated hemoglobin for reduction of virus and/or prion by heat treatment at suitable temperature with suitable concentration of antioxidant more than 5 mM;
g) performing reoxygenation of heat-treated deoxygenated hemoglobin composition to produce oxygenated hemoglobin composition;
h) optionally performing PEGylation of the heat treated oxygenated hemoglobin composition;
i) optionally performing carboxylation process to produce carboxylated PEGylated hemoglobin composition;
   wherein the oxygenated hemoglobin composition has lower charge variants in comparison to charge variants measured in oxygenated hemoglobin composition prepared by process with antioxidant at or less than 5 mM.

In some embodiments, the antioxidants are selected from N-acetyl cysteine, glutathione, ascorbic acid, preferably L-cysteine.

In some embodiments, the present invention provides a process for controlling and/or reducing formation of metHb during the manufacturing of oxygenated hemoglobin composition comprising:
a) performing deoxygenation of hemoglobin to form a deoxygenated hemoglobin;
b) heat treatment of the deoxygenated hemoglobin with about 5.5 mM to about 15 mM of L-cysteine;
c) reoxygenation of the deoxygenated hemoglobin to form an oxygenated hemoglobin composition;
d) measuring the metHb concentration in the oxygenated hemoglobin composition;
   wherein the oxygenated hemoglobin composition has less than 2% of metHb formation analyzed by co-oximetry;
e) optionally performing PEGylation of the oxygenated hemoglobin composition;
f) optionally performing carboxylation process to produce a carboxylated PEGylated hemoglobin composition;
   wherein the pegylated hemoglobin composition of step (e) or Pegylated carboxylated hemoglobin composition of step (f) maintains the metHb below 4%, preferably below 3%.

In some embodiments, the present invention provides a process for the preparation of stable hemoglobin composition comprising:
a) washing a fresh whole blood collected from animal sources to produce washed RBCs;
b) extracting a hemoglobin from the RBC's;
c) performing a filtration of the extracted hemoglobin;
d) performing ultrafiltration and concentration;
e) performing deoxygenation of ultrafiltered and concentrated hemoglobin;
f) performing viral inactivation of deoxygenated hemoglobin for reduction of virus and/or prion by heat treatment at suitable temperature;
g) performing reoxygenation of heat-treated deoxygenated hemoglobin to produce oxygenated hemoglobin composition;
h) optionally performing PEGylation of the heat treated deoxygenated hemoglobin;
i) optionally performing carboxylation process to produce carboxylated PEGylated hemoglobin composition;
   wherein the oxygenated hemoglobin composition has lower charge variants in comparison to charge variants measured in oxygenated hemoglobin composition prepared by process with antioxidant at or less than 5 mM.

In some embodiments, a hemoglobin composition comprising:
a) oxygenated hemoglobin;
b) metHb less than 9% of total oxygenated hemoglobin;
wherein the heat treatment is performed for about 10 hours.

In some embodiments, a hemoglobin composition comprising:
a) oxygenated hemoglobin;
b) metHb less than 4% of total oxygenated hemoglobin;
   wherein the heat treatment is performed for about 8 hours.

In some embodiments, the composition comprising an oxygenated hemoglobin that comprises main peak hemoglobin purity of more than 70%, less than 2% metHb, less than 25% charge variants, wherein the charge variants are acidic variant or basic variant of that main peak.

In some embodiments, the oxygenated hemoglobin is obtained from heat treated deoxyhemoglobin wherein the heat treatment is performed at least for 4 hours.

In some embodiments, the main peak purity, acidic and basic variants of oxygenated hemoglobin is analyzed by cIEF (capillary Iso Electric Focusing).

In some embodiments, the main peak purity of oxygenated hemoglobin is analyzed by cIEF (capillary Iso Electric Focusing) and metHb analyzed by co-oximetry.

In some embodiments, the main peak purity of oxygenated hemoglobin is more than 70%. In some embodiments, the main peak purity of oxygenated hemoglobin is about 70%, about 71%, about 72%, about 73%, about 74%, about 75%, about 76%, about 77%, about 78%, about 79%, about 80%, about 81%, about 82%, about 83%, about 84%, and about 85% or more.

In some embodiments, the charge variants in the reoxygenated/oxygenated hemoglobin composition are less than 25%. In some embodiments, the charge variants in the reoxygenated/oxygenated hemoglobin composition are acidic or basic charge variants. In some embodiments, the reoxygenated/oxygenated hemoglobin composition comprises less than 25%, less than 24%, less than 23%, less than 22%, less than 21%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5% or less charge variants and more than 60% of main peak purity.

In some embodiments, the acidic charge variants in the reoxygenated/oxygenated hemoglobin composition are less than 15%, less than 14%, less than 13%, less than 12%, less than 11%, less than 10%, less than 9%, less than 8%, less than 7%, less than 6%, less than 5% or less.

In some embodiments, the basic charge variants in the reoxygenated/oxygenated hemoglobin composition are less than 15%, less than 14%, less than 13%, less than 12%, less than 11%, less than 10%, less than 9%, less than 8%, less than 7%, less than 6%, less than 5% or less.

In some embodiments, the acidic and basic variants of oxygenated hemoglobin is analyzed by cIEF (capillary Iso Electric Focusing).

In some embodiments, the oxygenated hemoglobin has reduced viral load by more than 1 log₁₀.

In some embodiments, the oxygenated hemoglobin has reduced viral load selected from more than 1 log₁₀, more than 2 log₁₀, more than 3 log₁₀, more than 4 log₁₀ or more or more in comparison with the process performed without the heat treatment.

In some embodiments, the oxygenated hemoglobin has reduced prion load selected from more than 1 log₁₀, more than 2 log₁₀, more than 3 log₁₀, more than 4 log₁₀ or more or more in comparison with the process performed without the heat treatment.

In some embodiments, the L-cysteine suitable concentration is more than 5 mM. In some embodiments, the L-cysteine suitable concentration is selected from about 5.5 mM to about 20 mM.

In one aspect of such embodiment, the L-cysteine suitable concentration is selected from about 5.5 mM, about 5.6 mM, about 5.7 mM, about 5.8 mM, about 5.9 mM, about 6.0 mM, about 6.1 mM, about 6.2 mM, about 6.3 mM, about 6.4 mM, about 6.5 mM, about 6.6 mM, about 6.7 mM, about 6.8 mM, about 6.9 mM, about 7.0 mM, about 7.1 mM, about 7.2 mM, about 7.3 mM, about 7.4 mM, about 7.5 mM, about 7.6 mM, about 7.7 mM, about 7.8 mM, about 7.9 mM, about 8.0 mM, about 8.1 mM, about 8.2 mM, about 8.3 mM, about 8.4 mM, about 8.5 mM, about 8.6 mM, about 8.7 mM, about 8.8 mM, about 8.9 mM, about 9.0 mM, about 9.1 mM, about 9.2 mM, about 9.3 mM, about 9.4 mM, about 9.5 mM, about 9.6 mM, about 9.7 mM, about 9.8 mM, about 9.9 mM, about 10 mM, about 10.1 mM, about 10.2 mM, about 10.3 mM, about 10.4 mM, about 10.5 mM, about 10.6 mM, about 10.7 mM, about 10.8 mM, about 10.9 mM, about 11.0 mM, about 11.1 mM, about 11.2 mM, about 11.3 mM, about 11.4 mM, about 11.5 mM, about 11.6 mM, about 11.7 mM, about 11.8 mM, about 11.9 mM, about 12.0 mM, about 12.1 mM, about 12.2 mM, about 12.3 mM, about 12.4 mM, about 12.5 mM, about 12.6 mM, about 12.7 mM, about 12.8 mM, about 12.9 mM, about 13.0 mM, about 13.1 mM, about 13.2 mM, about 13.3 mM, about 13.4 mM, about 13.5 mM, about 13.6 mM, about 13.7 mM, about 13.8 mM, about 13.9 mM, about 14.0 mM, about 14.1 mM, about 14.2 mM, about 14.3 mM, about 14.4 mM, about 14.5 mM, about 14.6 mM, about 14.7 mM, about 14.8 mM, about 14.9 mM, about 15.0 mM, about 16.0 mM, about 16.5 mM, about 17.0 mM, about 17.5 mM, about 18.0 mM, about 18.5 mM, about 19.0 mM, about 19.5 mM, and about 20.0 mM.

In some embodiments, the heat treatment process is carried out at suitable temperature selected from about 55° C. to about 75° C.

In some embodiments, the heat treatment process is carried out at suitable temperature selected from about 57.0° C., about 57.1° C., about 57.2° C., about 57.3° C., about 57.4° C., about 57.5° C., about 57.6° C., about 57.7° C., about 57.8° C., about 57.9° C., about 58.0%, about 58.1° C., about 58.2° C., about 58.3° C., about 58.4° C., about 58.5° C., about 58.6° C., about 58.7° C., about 58.8° C., about 58.9° C., about 59.0%, about 59.1° C., about 59.2° C., about 59.3° C., about 59.4° C., about 59.5° C., about 59.6° C., about 59.7° C., about 59.8° C., about 59.9° C., about 60.0° C., about 60.1° C., about 60.2° C., about 60.3° C., about 60.4° C., about 60.5° C., about 60.6° C., about 60.7° C., about 60.8° C., about 60.9° C., about 61.0° C., about 61.1° C., about 61.2° C., about 61.3° C., about 61.4° C., about 61.5° C., about 61.6° C., about 61.7° C., about 61.8° C., about 61.9° C., about 62.0° C., about 62.1° C., about 62.2° C., about 62.3° C., about 62.4° C., about 62.5° C., about 62.6° C., about 62.7° C., about 62.8° C., about 62.9° C., about 63.0° C., about 63.1° C., about 63.2° C., about 63.3° C., about 63.4° C., about 63.5° C., about 63.6° C., about 63.7° C., about 63.8° C., about 63.9° C., about 64.0° C., about 64.1° C., about 64.2° C., about 64.3° C., about 64.4° C., about 64.5° C., about 64.6° C., about 64.7° C., about 64.8° C., about 64.9° C., and about 65.0° C.

In some embodiments, the heat treatment process is carried out at least more than 4 hours.

In some embodiments, the heat treatment process is carried out at a suitable time period selected from about 4 hours to about 15 hours.

In some embodiments, the heat treatment process is carried out at a suitable time period selected from about 5 hours to about 10 hours.

In some embodiments, the heat treatment process is performed for suitable time period selected from about 4 hours, about 4.1 hours, about 4.2 hours, about 4.3 hours, about 4.4 hours, about 4.5 hours, about 4.6 hours, about 4.7 hours, about 4.8 hours, about 4.9 hours, about 5.0 hours, about 5.1 hours, about 5.2 hours, about 5.3 hours, about 5.4 hours, about 5.5 hours, about 6.0 hours, about 6.1 hours, about 6.2 hours, about 6.3 hours, about 6.4 hours, about 6.5 hours, about 6.6 hours, about 6.7 hours, about 6.8 hours, about 6.9 hours, about 7.0, about 7.1 hours, about 7.2 hours, about 7.3 hours, about 7.4, about 7.5 hours, about 7.6 hours, about 7.7 hours, about 7.8 hours, about 7.9 hours, about 8.0 hours, about 8.1 hours, about 8.2 hours, about 8.3 hours, about 8.4 hours, about 8.5 hours, about 8.6 hours, about 8.7 hours, about 8.8 hours, about 8.9 hours, about 9.0 hours, about 9.1 hours, about 9.2 hours, about 9.3 hours, about 9.4 hours, about 9.5 hours, about 9.6 hours, about 9.7 hours, about 9.8 hours, about 9.9 hours, about 10.0 hours, about 10.1 hours, about 10.2 hours, about 10.3 hours, about 10.4 hours, about 10.5 hours, about 10.6 hours, about 10.7 hours, about 10.8 hours, about 10.9 hours, about 11.0 hours, about 11.1 hours, about 11.2 hours, about 11.3 hours, about 11.4 hours, about 11.5 hours, about 12.0 hours, about 12.5 hours, about 13.0 hours, about 13.5 hours, about 14.0 hours, about 14.5, and about 15.0 hours.

In some embodiments, the reoxygenated hemoglobin comprises total percentage of metHb less than 2%. In some embodiments, the reoxygenated hemoglobin comprises total percentage of metHb selected from about 0.5% to about 1.8% of total reoxygenated hemoglobin.

In some embodiments, the invention provides a reoxygenated hemoglobin comprises total percentage of metHb selected from about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, and about 1.8% of total reoxygenated hemoglobin.

In some embodiments, the stable hemoglobin composition is manufactured at large scale. In some embodiments, the invention provides a stable hemoglobin composition is manufactured at large scale more than 20 L.

In some embodiments, the stable hemoglobin composition is manufactured at large scale selected from about 20 L to about 1000 L.

In some embodiments, the large scale is more than 20 L. In certain embodiment, the large scale is more than 30 L, 50 L, 100 L, 200 L, 500 L, 1000 L, 5000 L.

In certain embodiment, oxygenated hemoglobin composition maintains metHIb below 2% during storage at 2-8° C. for more than 1 month, 3 months, 6 months, 9 months, 12 months, 15 months, 18 months, 21 months, 24 months, 27 months, 30 months, 33 months, 36 months, 39 months, 42 months, 45 months, and 48 months. In some embodiments, the p50 value of reoxygenated/oxygenated hemoglobin is 7-16 mmHg analyzed by Hemox Analyzer.

In some embodiments, the present invention provides a method to control metHb formation in the reoxygenated/oxygenated hemoglobin followed by viral inactivation of deoxygenated hemoglobin.

In some embodiments, the invention provides heat treatment process for eliminating or reducing virus in hemoglobin composition by using L-cysteine in suitable concentration more than 5 mM.

It is observed that 5 mM L-cysteine leads to the formation of metHb greater than 9% during heat treatment performed for about 9 hours to about 10 hours.

It is also observed that 5 mM L-cysteine leads to the formation of metHb greater than 4% during heat treatment performed for about 4 hours to about 5 hours.

In some embodiments, the invention performs reoxygenation of hemoglobin post heat treatment wherein the oxygenated hemoglobin maintains concentration of metHb is less than 2% analyzed by co-oximetry.

In some embodiments, the invention performs reoxygenation of hemoglobin post heat treatment wherein the oxygenated hemoglobin maintains concentration of metHb is less than 9% analyzed by co-oximetry when the heat treatment is performed for about 9 hours to about 10 hours with L-cysteine more than 5 mM.

In some embodiments, the invention performs reoxygenation of hemoglobin post heat treatment wherein the oxygenated hemoglobin maintains concentration of metHb is less than 4% analyzed by co-oximetry when the heat treatment is performed for about 4 hours to about 5 hours with L-cysteine more than 5 mM.

In some embodiments, the reoxygenation of the deoxyhemoglobin composition is performed by diafiltration. In some embodiments, the operating conditions for diafiltration (e.g., feed pressure, TMP, weight) are maintained by automated recipe in order to consistently remove L-cysteine from the viral inactivation process, reoxygenate the intermediate, and perform buffer exchange.

In some embodiments, reoxygenation of the deoxyhemoglobin composition is performed by maintaining the buffer temperature from about 5° C. to about 10° C.

In some embodiments, the reoxygenated hemoglobin composition is further proceeded for anion exchange purification by Sartobind Q filtration to produce purified hemoglobin.

In some embodiments, the purified hemoglobin of Sartobind Q filtrate is proceeded for PEGylation Process to produce PEGylated Hemoglobin with L-Cysteine.

In some embodiments, PEG-hemoglobin or PEG-Hb-CO composition comprises metHb less than 2%. In some embodiments, PEG-hemoglobin or PEG-Hb-CO composition comprises metHb about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, and about 1.8% of total PEG-Hb. It is observed that metHb is maintained below 3% during the long-term storage of PEG-Hb at 2-8° C. when the metHb is controlled below 2% during manufacturing of PEG-Hb hemoglobin. In certain embodiment, PEG-hemoglobin or PEG-Hb-CO composition maintains metHb below 2% during storage at 2-8° C. more than 1 month, 3 months, 6 months, 9 months, 12 months, 15 months, 18 months, 21 months, 24 months, 27 months, 30 months, 33 months, 36 months, 39 months, 42 months, 45 months, and 48 months.

In some embodiments, the Pegylated hemoglobin or Pegylated carboxylated hemoglobin maintains the metHb below 4%, preferably below 3%.

In some embodiments, the Pegylated hemoglobin or Pegylated carboxylated hemoglobin maintains the metHb below 4% preferably below 3% and substantially free of virus and prions.

In some embodiments, the Pegylated hemoglobin or Pegylated carboxylated hemoglobin maintains acidic charge variants less than 12%, less than 11%, less than 10%, less than 9%, less than 8%, less than 7%, less than 6%, less than 5% or less; basic charge variants less than 12%, less than 11%, less than 10%, less than 9%, less than 8%, less than 7%, less than 6%, less than 5% or less.

In some embodiments, the present invention provides a method of treating a condition that can be ameliorated by oxygenating the red blood cells of a patient in need of such treatment, by administering to patient a therapeutically effective amount of stable hemoglobin composition comprising:
a) oxygenated hemoglobin comprising main peak purity not less than 70%;
b) one or more impurity selected from metHb, acidic variants, basic variants, virus particles and/or prions;
wherein the oxygenated hemoglobin comprising not more than 2% metHb of total oxygenated hemoglobin.

In some embodiments, the present invention provides a method of treating a condition that can be ameliorated by oxygenating the red blood cells of a patient in need of such treatment, by administering to patient a therapeutically effective amount of stable hemoglobin composition comprising:
a) oxygenated hemoglobin comprising not more than 2% metHb;
b) oxygenated hemoglobin comprising main peak purity not less than 70%;
c) oxygenated hemoglobin comprising less than 12% acidic variants of the main peak;
d) oxygenated hemoglobin comprising less than 11% basic variants of the main peak;
e) oxygenated hemoglobin comprising substantially free of virus particles and/or prion.

In some embodiments, the present invention provides the stable composition of hemoglobin comprising:
a) reoxygenated hemoglobin containing not more than 2% MetHb; and
b) reoxygenated hemoglobin main peak purity not less than 60%,
wherein, MetHb is analyzed by co-oximetry and reoxygenated hemoglobin main peak purity is analyzed by cIEF (capillary Iso Electric Focusing).

In some embodiments, the invention provides heat treatment process for eliminating or reducing virus in hemoglobin composition by using L-cysteine in suitable concentration.

In some embodiments, the L-cysteine suitable concentration is more than 5 mM.

In an embodiment the L-cysteine suitable concentration is selected from about 5.5 mM to about 15 mM.

In one aspect of such embodiment, the L-cysteine suitable concentration is selected from about 5.5 mM, about 5.6 mM. about 5.7 mM, about 5.8 mM, about 5.9 mM, about 6.0 mM, about 6.1 mM, about 6.2 mM, about 6.3 mM, about 6.4 mM, about 6.5 mM, about 6.6 mM, about 6.7 mM, about 6.8 mM, about 6.9 mM, about 7.0 mM, about 7.1 mM, about 7.2 mM, about 7.3 mM, about 7.4 mM, about 7.5 mM, about 7.6 mM, about 7.7 mM, about 7.8 mM, about 7.9 mM, about 8.0 mM, about 8.1 mM, about 8.2 mM, about 8.3 mM, about 8.4 mM, about 8.5 mM, about 8.6 mM, about 8.7 mM, about 8.8 mM, about 8.9 mM, about 9.0 mM, about 9.1 mM, about 9.2 mM, about 9.3 mM, about 9.4 mM, about 9.5 mM, about 9.6 mM, about 9.7 mM, about 9.8 mM, about 9.9 mM, about 10 mM, about 10.1 mM, about 10.2 mM, about 10.3 mM, about 10.4 mM, about 10.5 mM, about 10.6 mM, about 10.7 mM, about 10.8 mM, about 10.9 mM, about 11.0 mM, about 11.1 mM, about 11.2 mM, about 11.3 mM, about 11.4 mM, about 11.5 mM, about 11.6 mM, about 11.7 mM, about 11.8 mM, about 11.9 mM, about 12.0 mM, about 12.1 mM, about 12.2 mM, about 12.3 mM, about 12.4 mM, about 12.5 mM, about 12.6 mM, about 12.7 mM, about 12.8 mM, about 12.9 mM, about 13.0 mM, about 13.1 mM, about 13.2 mM, about 13.3 mM, about 13.4 mM, about 13.5 mM, about 13.6 mM, about 13.7 mM, about 13.8 mM, about 13.9 mM, about 14.0 mM, about 14.1 mM, about 14.2 mM, about 14.3 mM, about 14.4 mM, about 14.5 mM, about 14.6 mM, about 14.7 mM, about 14.8 mM, about 14.9 mM, about 15.0 mM. 6.0 mM, about 6.1 mM, about 6.2 mM, about 6.3 mM, about 6.4 mM, about 6.5 mM, about 6.6 mM, about 6.7 mM, about 6.8 mM, about 6.9 mM, about 7.0 mM, about 7.1 mM, about 7.2 mM, about 7.3 mM, about 7.4 mM, about 7.5 mM, about 7.6 mM, about 7.7 mM, about 7.8 mM, about 7.9 mM, about 8.0 mM, about 8.1 mM, about 8.2 mM, about 8.3 mM, about 8.4 mM, about 8.5 mM, about 8.6 mM, about 8.7 mM, about 8.8 mM, about 8.9 mM, about 9.0 mM, about 9.1 mM, about 9.2 mM, about 9.3 mM, about 9.4 mM, about 9.5 mM, about 9.6 mM, about 9.7 mM, about 9.8 mM, about 9.9 mM, about 10 mM, about 10.1 mM, about 10.2 mM, about 10.3 mM, about 10.4 mM, about 10.5 mM, about 10.6 mM, about 10.7 mM, about 10.8 mM, about 10.9 mM, about 11.0 mM, about 11.5 mM, about 12.0 mM, about 12.5 mM, about 13.0 mM, about 13.5 mM, about 14.0 mM, about 14.5 mM, about 15.0 mM.

In some embodiments, the invention provides a stable hemoglobin composition comprising;
a. performing the heat treatment of deoxyhemoglobin composition at suitable temperature for suitable period of time;
b. maintains suitable concentration of antioxidant more than 5 mM;
c. reoxygenation of the hemoglobin composition;
d. measuring the MetHb concentration in reoxygenated hemoglobin composition,
   wherein the reoxygenated hemoglobin composition has lower concentration of the MetHb in comparison to MetHb concentration measured in reoxygenated hemoglobin composition treated with antioxidant at or less than 5 mM.

In some embodiments, the invention performs reoxygenation of hemoglobin post heat treatment wherein the hemoglobin maintains concentration of MetHb is below 2% analyzed by cooximetry.

In some embodiments, the percentage of MetHb available in reoxygenated hemoglobin solution during or post heat treatment selected from about less than 2%, about less than 1.9%, about less than 1.8%, about less than 1.7%, about less than 1.6%, about less than 1.5%, about less than 1.4%, about less than 1.3%, about less than 1.2%, about less than 1.1%, about less than 1.0%.

In some embodiments, the heat treatment process is carried out at suitable temperature selected from above 59° C. to about 65° C. In some embodiments, the heat treatment process is carried out at suitable temperature selected from about 60.0° C., about 60.1° C., about 60.2° C., about 60.3° C., about 60.4° C., about 60.5° C., about 60.6° C., about 60.7° C., about 60.8° C., about 60.9° C., about 61.0° C., about 61.1° C., about 61.2° C., about 61.3° C., about 61.4° C., about 61.5° C., about 61.6° C., about 61.7° C., about 61.8° C., about 61.9° C., about 62.0° C., about 62.1° C., about 62.2° C., about 62.3° C., about 62.4° C., about 62.5° C., about 62.6° C., about 62.7° C., about 62.8° C., about 62.9° C., about 63.0° C., about 63.1° C., about 63.2° C., about 63.3° C., about 63.4° C., about 63.5° C., about 63.6° C., about 63.7° C., about 63.8° C., about 63.9° C., about 64.0° C., about 64.1° C., about 64.2° C., about 64.3° C., about 64.4° C., about 64.5° C., about 64.6° C., about 64.7° C., about 64.8° C., about 64.9° C., about 65.0° C.

In some embodiments, the heat treatment process is carried out at suitable time period selected from about 4 hours to about 15 hours. In some embodiments, the heat treatment process is performed for suitable time period selected from about 4 hours, about 4.1 hours, about 4.2 hours, about 4.3 hours, about 4.4 hours, about 4.5 hours, about 4.6 hours, about 4.7 hours, about 4.8 hours, about 4.9 hours, about 5 hours, about 5.1 hours, about 5.2 hours, about 5.3 hours, about 5.4 hours, about 5.5 hours, about 6.0 hours, about 6.1 hours, about 6.2 hours, about 6.3 hours, about 6.4 hours, about 6.5 hours, about 6.6 hours, about 6.7 hours, about 6.8 hours, about 6.9 hours, about 7.0, about 7.1 hours, about 7.2 hours, about 7.3 hours, about 7.4, about 7.5 hours, about 7.6 hours, about 7.7 hours, about 7.8 hours, about 7.9 hours, about 8.0 hours, about 8.1 hours, about 8.2 hours, about 8.3 hours, about 8.4 hours, about 8.5 hours, about 8.6 hours, about 8.7 hours, about 8.8 hours, about 8.9 hours, about 9.0 hours, about 9.1 hours, about 9.2 hours, about 9.3 hours, about 9.4 hours, about 9.5 hours, about 9.6 hours, about 9.7 hours, about 9.8 hours, about 9.9 hours, about 10.0 hours, about 10.1 hours, about 10.2 hours, about 10.3 hours, about 10.4 hours, about 10.5 hours, about 10.6 hours, about 10.7 hours, about 10.8 hours, about 10.9 hours, about 11.0 hours, about 11.1 hours, about 11.2 hours, about 11.3 hours, about 11.4 hours, about 11.5 hours, about 12.0 hours, about 12.5 hours, about 13.0 hours, about 13.5 hours, about 14.0 hours, about 14.5, about 15.0 hours.

In some embodiments, the invention can be applied to eliminate or reduce all virus forms, particularly DNA and RNA viruses, enveloped and unenveloped viruses, furthermore to virions and prions or other similar biological systems and also bacteria and fungi.

The method is preferably used to reduce the Bovine viral diarrheal virus (BVDV) virus, Retrovirus, Parvovirus contamination, Bovine spongiform encephalopathy (BSE), and Transmissible spongiform encephalopathies (TSEs) contamination from the hemoglobin solution.

In some embodiments, the invention provides a stable hemoglobin composition comprising:
a) reoxygenated Hb purity more than 60%;
b) reoxygenated Hb substantially free of virus particles; c) reoxygenated Hb substantially free from prions.

In some embodiments, the purity of reoxygenated Hb is about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85% or more.

In some embodiments, viral reduction of heat-treated hemoglobin solution has been reduced by a factor of more than 1 log₁₀, more than 2 log₁₀, more than 3 log₁₀, more than 4 log 10 or more in comparison with before the heat treatment.

In some embodiments, the invention provides a process for the preparation of stable hemoglobin composition comprising;
a. performing the heat treatment of deoxyhemoglobin composition at suitable temperature for suitable period of time;
b. maintains suitable concentration of antioxidant more than 5 mM;
c. reoxygenation of hemoglobin composition;
d. measuring the MetHb concentration in reoxygenated hemoglobin composition,
   wherein the reoxygenated hemoglobin composition has lower concentration of the MetHb in comparison to MetHb concentration measured in reoxygenated hemoglobin composition prepared by process with antioxidant at or less than 5 mM.

In certain embodiment the invention provides a process for the preparation of stable hemoglobin composition comprising;
a. performing the heat treatment of deoxyhemoglobin composition at suitable temperature for suitable period of time;
b. maintains suitable concentration of antioxidant more than 5 mM;
c. reoxygenation of the hemoglobin composition;
d. measuring the charge variants in the reoxygenated hemoglobin composition;
   wherein the reoxygenated hemoglobin composition has lower charge variants in comparison to charge variants measured in reoxygenated hemoglobin composition prepared by process with antioxidant at or less than 5 mM.

In some embodiments, the condition of patient is selected from the group comprising acute respiratory distress syndrome, bronchiectasis, bronchopulmonary dysplasia, chronic obstructive pulmonary disease, cystic fibrosis, emphysema, lymphangiomatosis, primary ciliary dyskinesia, cancer, tumour, cancers of the lung, pulmonary hypertension, pulmonary fibrosis, pulmonary vascular disease, pulmonary sarcoidosis, pneumonia and bronchitis, infectious diseases that affect the lung's ability to transport oxygen.

In some embodiments, the said condition is selected from the group comprising acute respiratory distress syndrome, bronchiectasis, bronchopulmonary dysplasia, chronic obstructive pulmonary disease, cystic fibrosis, emphysema, lymphangiomatosis, primary ciliary dyskinesia, cancer, tumor, cancers of the lung, pulmonary hypertension, pulmonary fibrosis, pulmonary vascular disease, pulmonary sarcoidosis, pneumonia and bronchitis, infectious diseases that affect the lung's ability to transport.

In some embodiments, the said condition is an anemia selected from the group consisting of blood loss anemias, anemias caused by faulty red blood cell production, anemias caused by red blood cell destruction, and a combination thereof, wherein the cancers are selected from solid tumors, soft tissue carcinoma, lung cancer, bone cancer, metastatic cancer, adrenal cancer, anal cancer, appendix cancer, bile duct cancer, bladder cancer, bone cancer, brain cancer, breast cancer.

In some embodiments, the present invention provides a method of administration of a stable hemoglobin composition comprising:
a) oxygenated hemoglobin comprising main peak purity not less than 70%;
b) one or more impurity selected from metHb, acidic variants, basic variants, virus particles and/or prions;
wherein the oxygenated hemoglobin comprising not more than 2% metHb of total oxygenated hemoglobin.

In some embodiments, the present invention provides a method of administration of a stable hemoglobin composition comprising:
a) oxygenated hemoglobin comprising main peak purity not less than 70% and one or more impurity comprising;
b) oxygenated hemoglobin comprising not more than 2% metHb;
c) oxygenated hemoglobin comprising less than 12% acidic variants of the main peak;
d) oxygenated hemoglobin comprising less than 11% basic variants of the main peak;
e) oxygenated hemoglobin comprising substantially free of virus particles and/or prion;
   wherein, metHb is analyzed by co-oximetry and reoxygenated/oxygenated hemoglobin main peak purity is analyzed by cIEF (capillary Iso Electric Focusing).

In some embodiments, the dosing frequency is once a daily or twice a daily.

In some embodiments, the oxygenated hemoglobin is pharmaceutically stable.

The present invention surprisingly found that the concentration of L-cysteine greater than 5 mM can effectively control one or more metHb formation, charge variant and hemoglobin purity in oxyhemoglobin state (reoxygenated hemoglobin) during manufacturing/purification.

The hemoglobin composition provides stability of the hemoglobin composition from batch to batch and thereby helps to achieve batch to batch consistency. To provide further clarity, stable hemoglobin composition maintains main purity peak of hemoglobin/reoxygenated hemoglobin at desired level at more than 70% and metHb less than 2% which is also helpful in long term storage of PEG-hemoglobin or PEG-Hb-CO composition.

In certain embodiment, oxygenated hemoglobin composition maintains metHb below 2% more than 1 month, 3 months, 6 months, 9 months, 12 months, 15 months, 18 months, 21 months, 24 months, 27 months, 30 months, 33 months, 36 months, 39 months, 42 months, 45 months, and 48 months.

In some embodiments, the oxygenated hemoglobin is obtained after post heat treatment of deoxygenated hemoglobin.

In some embodiments, the invention includes a method of synergistically treating inflammation, vasoconstriction and hypoxia in a patient in need of such treatment, by administering a therapeutically effective amount of an HBOC composition in accordance with the invention.

In some embodiments, the present invention also provides methods of treating trauma, shock, ischemia and any other illness that is amenable to amelioration by enhancing the oxygen or carbon monoxide content of tissues or organs. Compositions of the invention rapidly restore tissue oxygenation and fully repay oxygen debt in animal models of severe traumatic shock in which at least 50% of subjects normally die from hemorrhagic shock. Utilizing an exemplary formulation, a single unit of a composition of the invention repays the oxygen debt to all the major organs, opens the microvasculature and restores Mean Arterial Pressure.

In an exemplary embodiment, the method includes administering to the subject an amount of a composition of any of the invention sufficient to accomplish the delivery of oxygen or carbon monoxide to one or more tissue and/or organ.

In various embodiments, the invention provides a method of reversing oxygen debt in a member selected from tissues and organs of a subject suffering from hemorrhagic shock. In an exemplary embodiment, the method includes administering to the subject an amount of a composition of any of the invention sufficient to reverse the oxygen debt. A similar method is provided for increasing the carbon monoxide content of a tissue, whether in response to a loss in carbon monoxide content due to disease, injury, etc., or as a means to gain therapeutic benefits from increasing carbon monoxide content in the tissue over the normal levels found in the tissue in a healthy or disease state.

In various embodiments, the invention provides a method of inducing angiogenesis in the tissues of a subject by administering to the subject an amount of a composition of the invention effective to induce angiogenesis. In exemplary embodiments, angiogenesis is induced in tissues suffering from oxygen deficiency. In further exemplary embodiments, the tissues or organs in which angiogenesis is induced are tissues or organs of a subject suffering from oxygen deficiency. In an exemplary embodiment, the method includes administering to the subject an amount of a composition of any of the invention sufficient to reverse the oxygen deficiency.

The present examples given below are illustrative purpose and scope of the invention should not be considered limiting to them.

### Example 1

### Extraction and Purification of Hemoglobin

This first step involves washing the blood cells free of plasma. On the bench scale this was accomplished by repetitive washing, centrifugation and decanting. This process appears more efficient than the vertical continuous flow centrifuge.

The red cells are washed 4× with buffer (1.2% NaCl with 10 mM phosphate, pH 7.8). The red cells are then lysed (the hemoglobin is extracted without true cell lysis) by the slow addition of 1.5 volumes of WFI over a period of 2 hours. The status of lysis (again, hemoglobin extraction) is monitored by sampling conductivity until it is in the range of 5.50-7.00 μS. The % Hb is determined using a Radiometer OSM3 Hemoximeter. The Osmolality is also tested and is in the range of 130-150 mOsmol. Once hemoglobin extraction is complete the content is pressure filtered through a 1 μm cellulose based depth filter, followed by 0.45/0.2 μm sterile polysulfone based filter into a second tank which serves as the reservoir for the next step. The hemoglobin solution is then pumped through a 300 Kd ultrafilter into a jacketed tank at 10° C., which serves as the initial viral removal step and removes high molecular weight proteins.

The hemoglobin is concentrated using a 10 KD MWCO system.

### Deoxygenation Process to Produce Deoxygenated Hemoglobin

The Concentrated Hemoglobin is then deoxygenated by passage through a counter-current membrane contactor. As the Concentrated Hemoglobin is pumped through the shell of the device, argon gas is pumped into the core in the opposite direction. The membrane in the device is permeable to gases only. The intermediate is circulated through the device from the intermediate tank until the oxygen content of the hemoglobin solution (HbO2) is <10.0%, 7.5 mM-15 mM L-cysteine is then added to the intermediate creating Deoxygenated Hemoglobin.

The Hb eluate can be deoxygenated by gas transfer of an inert gas across a phase membrane. Such inert gases include, for example argon. It is understood that other means for deoxygenating a solution of hemoglobin, which are known in the art, can be used to deoxygenate the Hb eluate.

Deoxygenation continues until the pO2 of the Hb solution is reduced to a desired level, for example wherein the oxygenated Hb (oxyhemoglobin or HbO2) content in the Hb solution is about 10% or less, 5% or less 3% or less or 1% or less.

### Viral Inactivation Process to Produce Hemoglobin Post Viral Inactivation

The Viral Inactivation process is initiated following deoxygenation and involves heat treatment to inactivate adventitious agents. Deoxygenated Hemoglobin with L-cysteine is required during the inactivation process; residual oxyhemoglobin will denature or precipitate. The Deoxygenated Hemoglobin is heated and held at about 60° C. for 10 hours before being cooled again. After treatment, the Hemoglobin Post Viral Inactivation is filtered through a 0.45 μm/0.2 μm capsule filter. For deoxygenated hemoglobin, liquid temperatures above 65° C. may lead to methemoglobin formation in the intermediate product (indicating degraded hemoglobin) while also approaching the denaturation (thermal melting) temperature for bovine deoxyhemoglobin. In order to achieve the critical adventitious agent log reduction, a temperature above 59° C. must be held for a minimum of two hours.

### Pre-PEG Diafiltration Process to Produce Diafiltered Reoxygenated Hemoglobin (Reoxygenation of Hemoglobin)

The in-process critical quality attribute for this unit operation is % HbO2. The % HbO2 is critical for control prior to hemoglobin PEGylation since its conformation within the deoxygenated state changes and would subsequently impact the PEGylation reaction. Additionally, operating conditions (e.g., feed pressure, TMP, weight) are maintained by automated recipe in order to consistently remove L-cysteine from the viral inactivation process, reoxygenate the intermediate, and perform buffer exchange. Co-oximetry (% HbO2), cIEF, pH, and osmolality measurements are performed to confirm buffer exchange completion to the desired product quality necessary for hemoglobin PEGylation.

### PEGylation Process to Produce PEGylated Hemoglobin with L-Cysteine

Prior to PEGylation, the reoxygenated hemoglobin is subjected to adventitious agent removal filtration. PEGylation process is the conjugation of eight to ten chains of 5 kD activated polyethylene glycol (SC-PEG-5K) molecules to the purified bovine hemoglobin molecule by a controlled method described below. The Purified Hemoglobin is PEGylated using a specified molar ratio of PEG to hemoglobin added at a specified rate. The reaction is allowed to proceed to completion (for a specified time and indicated by a stable pH). During the PEG addition and subsequent period of time until the reaction pH has stabilized, 1 N NaOH is added directly to the reaction mixture to maintain a pH range of 7.95-8.20. When the intermediate pH is stable, 15 mM L-cysteine is added for control of % metHb formation and quench the PEGylation reaction overnight. The PEG-Hb with L-cysteine is then 0.45 μm/0.2 μm filtered into a stainless-steel tank.

### Post-PEG Diafiltration Process to Produce Purified PEGylated Hemoglobin (DS)

Using a 50 kD MWCO diafiltration system, the Filtered PEGylated Hemoglobin is diafiltered with FFB at ten times the intermediate volume. Impurities removed by diafiltration include N-hydroxysuccinimide (NHS), free PEG, unreacted bovine hemoglobin and L-cysteine due to the MWCO of the diafiltration membrane. After the diafiltration, the hemoglobin is concentrated to a final tHb (total hemoglobin) of 3.6-4.6 g % tHb before the Diafiltered PEGylated Hemoglobin is 0.45 μm/0.2 μm capsule filtered and transferred out of the stainless-steel skid into the final single-use bag. At this stage, the Purified PEGylated Hemoglobin Bulk is the bulk drug substance.

### Carboxylation Process to Produce Carboxylated PEGylated Hemoglobin with Excipients

Starting with the Purified PEGylated Hemoglobin Bulk (drug substance bulk), carbon monoxide is sparged into the bulk at 3-7 liters per minute until the HbCO concentration is ≥98%.

The final excipients are L-cysteine and dextrose, are dissolved within FFB, 0.45/0.2 μm filtered and dispensed into the Carboxylated PEGylated Hemoglobin at 0.61 g/kg and 5.0 g/kg, respectively. The pH of the L-cysteine solution is adjusted to 8.1-8.2 before the excipient solutions are combined. Filtered excipient solutions are added to the carboxylated PEGylated intermediate to prepare Carboxylated PEGylated Hemoglobin with Excipients. Carboxylation is re-initiated to remove excess oxygen that may have been supplied to the carboxylated intermediate through excipient solution addition. The Carboxylated PEGylated Hemoglobin with Excipients (formulated drug substance) is overlaid with argon to minimize exposure to oxygen and is maintained at 2-8° C. prior to filling. Osmolality (310-360 mOsm/kg), pH (7.8-8.2), g % tHb (3.8-4.2), and % HbCO (≥98%) are the final product ranges for production of the final product.

### Example 2: Process to Generate Purified Reoxygenated Hemoglobin

### Extraction of Hemoglobin

The red cells are washed 4× with phosphate buffer (1.2% NaCl with 10 mM phosphate, pH 7.8). The red cells are then lysed (the hemoglobin is extracted without true cell lysis) by the slow addition of WFI. The percentage Hb is determined using a Radiometer OSM3 Hemoximeter. Once hemoglobin extraction is completed, the content is pressure filtered through a 1 μm cellulose based depth filter, followed by 0.45/0.2 μm sterile polysulfone based filter into a second tank which serves as the reservoir for the next step. The hemoglobin solution is then pumped through a 300 Kd ultrafilter into a jacketed tank at 10° C.

The hemoglobin is concentrated using a 10 KD MWCO system to get approximately 16 g % concentration and then deoxygenated by recirculation through a hollow fiber membrane contactor until the hemoglobin solution has less than 10% HbO2.

### Heat Treatment of Hemoglobin

Deoxygenated and concentrated hemoglobin is transferred to a jacketed vessel and L-cysteine solution is added to achieve ≥5 mM concentration. The L-cysteine acts as a reducing agent/oxygen quenching agent and prevents oxidation of hemoglobin into methemoglobin while the L-cysteine gets consumed in the process. It is known the clearance of viruses is directly proportional to the duration and temperature of the heat treatment.

Table 1 shows clearance of prions from hemoglobin solution when heat-treated at different duration. There is a linear increase in clearance as the duration of heat-treatment is increased from 1 hour to 4 hours.

TABLE 1
Detailed Initial load, Output load, and Log 10 reduction of Transmissible
spongiform encephalopathies (TSEs)/Prion by heat treatment of
deoxygenated hemoglobin. ¹PWBU = Prion Western Blot Unit

| | Initial Load | Output load | |
|---|---|---|---|
| | (log₁₀ | (log₁₀ | Log₁₀Reduction |
| Step | PWBU)¹ | TCID₅₀) | (LRV) |
| Run no. 1 Hold 60° C. | 6.94 | 5.94 | 1.00 |
| T = 0 hr | | | |
| Run no. 1 Hold 60° C. | 6.94 | 5.94 | 1.00 |
| T = 1 hr | | | |
| Run no. 1 Hold 60° C. | 6.94 | 5.44 | 1.50 |
| T = 2 hr | | | |
| Run no. 1 Hold 60° C. | 6.94 | 4.94 | 2.00 |
| T = 4 hr | | | |

Hence, for every therapeutic protein the longest duration that does not denature the protein is typically chosen. In this example, the temperature is increased to 60±1° C. and the solution is stirred gently for up to 10 hours to carry out heat inactivation of viruses and prions. Post heat treatment, the solution is rapidly cooled to refrigerated temperatures. Heat treated deoxyhemoglobin is then filtered through 0.45/0.2 μm sterile polysulfone based filter.

### Purification and Reoxygenation

Heat treated deoxyhemoglobin is filtered using a 10 KD MWCO system to generate purified hemoglobin (free of L-cysteine). During this step hemoglobin is exposed to oxygenated buffer and purified hemoglobin is predominantly (90% or more) in oxyhemoglobin form. The metHb is controlled to be approximately 2% or below.

### Example 3

### L-Cysteine Concentration and Hemoglobin Purity

In this example, post deoxygenation step, the solution was divided into 4 aliquots A through D. The aliquots were spiked with varying concentrations (5 mM, 7.5 mM, 10 mM, and 15 mM) of L-cysteine. Subsequently, the samples were subjected to 10 hours of heat treatment at 60° C. During the process of heat treatment, a small test sample was taken out from each reaction mixture at 0 hours, 4 hours, 6 hours, 8 hours, and 10 hours. Before the next step, all these heat-treated samples were analyzed for methemoglobin by co-oximetry and hemoglobin purity by capillary Iso Electric Focusing (cIEF) method.

Next, all of these samples were washed with more than 10× volume of 1% buffered saline (10 kDa filter) to generate purified oxyhemoglobin. Resulting hemoglobin was analyzed for methemoglobin by co-oximetry. The following table shows the effect of L-cysteine concentration on metHb content in purified hemoglobin. For each concentration of L-cysteine, 10-hour heat treatment showed highest metHb value (around 2%). However, 5 mM L-cysteine failed to control metHb post reoxygenation step when the heat treatment was more than 8 hours. The metHb increased to be as high as 10%.

TABLE 2
Percentage of metHb in purified oxyhemoglobin
samples generated using different L-cysteine
concentrations and heat treatment duratins.

| L-Cysteine Concentration | | Heat Duration (hr) | | | | |
|---|---|---|---|---|---|---|
| (mM)↓ | | 0 hr | 4 hr | 6 hr | 8 hr | 10 hr |
| 5 | mM | 1.1 | 0.0 | 1.5 | 4.3 | 9.9 |
| 7.5 | mM | 1.1 | 0.0 | 0.9 | 1.5 | 1.9 |
| 10 | mM | 1.1 | 1.1 | 1.2 | 1.3 | 1.8 |
| 15 | mM | 0.9 | 1.2 | 1.3 | 1.4 | 1.9 |

### Example 4

### L-Cysteine Concentration and Hemoglobin Purity

The co-oximetry analysis for tHB, percentage of metHB, percentage of HbCO, percentage of DeoxyHb and percentage of HbO2 was performed on undiluted samples using OSM3 co-oximeter by Radiometer.

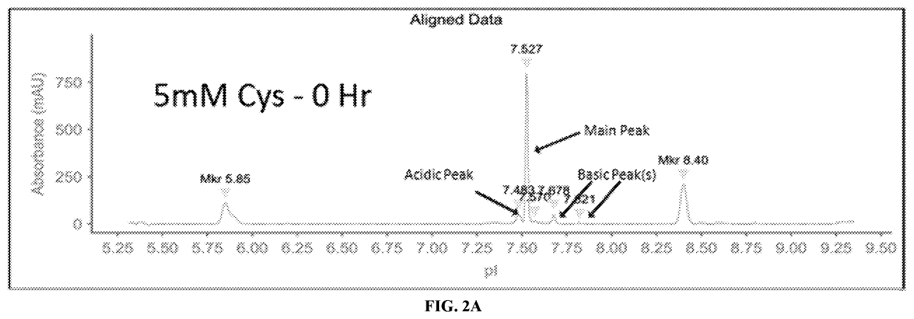

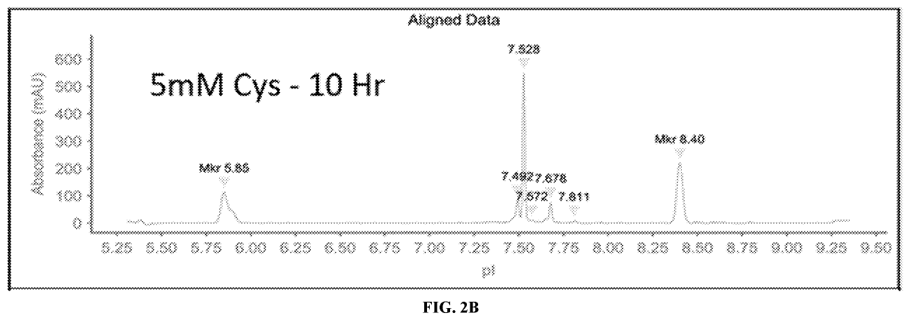

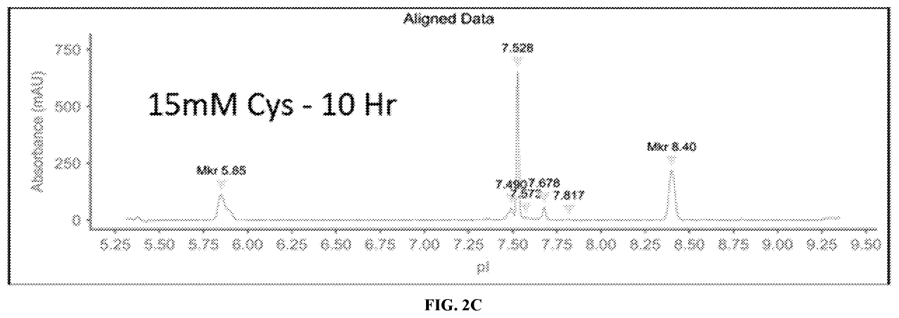

The hemoglobin charge purity by cIEF was determined to identify ability of L-cysteine to prevent oxidative damage on hemoglobin and effect of L-cysteine concentration was studied. Briefly, samples were diluted to 1 mg/mL and analyzed on Maurice platform. Charge profiles of the hemoglobin samples with 5 mM L-cysteine at 0 hr (baseline) and 10 hr in comparison with 15 mM L-cysteine at 10 hr is disclosed in FIG. 2. FIG. 2 shows cIEF (Maurice) profiles of a) typical hemoglobin pre-heat treatment in presence of 5 mM L-cysteine. b) after treatment at 60° C. for 10 hours in presence of 5 mM L-cysteine showing elevation in acidic and basic charge variants. c) after treatment at 60° C. for 10 hours in presence of 15 mM L-cysteine showing no elevation in charge variants shown in table 3.

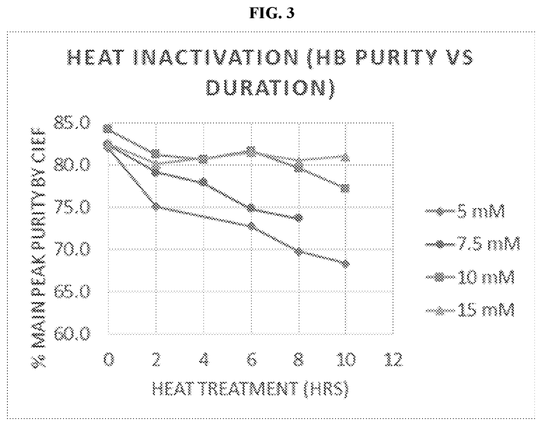

Across samples, increase in acidic and basic variants levels mostly followed the trend observed in post purification metHb levels i.e., higher metHb corresponded to reduced main peak purity. 5 mM L-cysteine samples treated up to 10 hours dropped main peak purity below 70%, while-10 mM and 15 mM L-cysteine showed almost no decline in hemoglobin main peak purity (above 75%). FIG. 3 compares the main peak purity at different L-cysteine concentrations vs heat duration.

| | | Heat Duration (hr) | | | | | | | | | |
|---|---|---|---|---|---|---|---|---|---|---|---|
| L-Cysteine | | 0 hr | | 4 hrs | | 6 hrs | | 8 hrs | | 10 hrs | |
| Concentration | | Acidic | Basic | Acidic | Basic | Acidic | Basic | Acidic | Basic | Acidic | Basic |
| (mM) | | (%) | (%) | (%) | (%) | (%) | (%) | (%) | (%) | (%) | (%) |
| 5 | mM | 9.1 | 6.2 | 15.9 | 10.9 | 14.3 | 9.5 | 15.1 | 11.5 | 15.5 | 12.3 |
| 7.5 | mM | 9.0 | 6.0 | 10.9 | 8.6 | 12.0 | 9.0 | 12.1 | 10.7 | 8.9 | 8.2 |
| 10 | mM | 7.9 | 5.6 | 9.7 | 6.8 | 8.7 | 7.0 | 9.9 | 7.7 | 10.9 | 8.8 |
| 15 | mM | 9.0 | 5.8 | 11.9 | 7.1 | 9.0 | 6.8 | 9.0 | 7.7 | 8.3 | 8.0 |

### EQUIVALENTS

While specific embodiments of the present disclosure have been discussed, the above specification is illustrative and not restrictive. Many variations of the present disclosure will become apparent to those skilled in the art upon review of this specification. The full scope of the disclosure should be determined by reference to the claims, along with their full scope of equivalents, and the specification, along with such variations.

We claim:

1. A hemoglobin composition comprising:
a) an oxygenated hemoglobin;
b) a metHb less than 2% of total oxygenated hemoglobin.

2. The hemoglobin composition according to claim 1, comprising:
a) An oxygenated hemoglobin comprising main peak purity not less than 70%;
b) one or more impurity selected from metHb, charge variants selected from acidic variants, basic variants and optionally virus particles and/or prions;
wherein the oxygenated hemoglobin comprises not more than 2% metHb of total oxygenated hemoglobin.

3. The composition according to claim 2, wherein the oxygenated hemoglobin comprises total percentage of metHb selected from about 0.5% to about 1.8% of total oxygenated hemoglobin.

4. The composition according to claim 3, wherein the oxygenated hemoglobin comprises total percentage of metHb selected from about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8% of total oxygenated hemoglobin.

5. The composition according to claim 2, wherein the stable hemoglobin composition is manufactured at large scale.

6. The composition according to claim 2, wherein the main peak purity of oxygenated hemoglobin is about 70%, about 71%, about 72%, about 73%, about 74%, about 75%, about 76%, about 77%, about 78%, about 79%, about 80%, about 81%, about 82%, about 83%, about 84%, about 85% or more.

7. The composition according to claim 2, wherein the charge variants are below 25%.

8. The composition according to claim 2, wherein the acidic variants are less than about 12%, less than about 11%, less than about 10%, less than about 9%, less than about 8%, less than about 7% or less; wherein the basic variants are less than about 11%, less than about 10%, less than about 9%, less than about 8%, less than about 7% or less.

9. The composition according to claim 1, wherein the oxygenated hemoglobin is obtained post heat treatment of deoxygenated hemoglobin.

10. The composition according to claim 2, wherein the oxygenated hemoglobin has reduced viral and/or prion load by more than 1 log₁₀.

11. The composition according to claim 10, wherein the oxygenated hemoglobin has reduced viral and/or prion load selected from more than 1 log₁₀, more than 2 log₁₀, more than 3 log₁₀, more than 4 log 10 or more.

12. The composition according to claim 2, wherein the composition of oxygenated hemoglobin comprising:
a) oxygenated hemoglobin comprising main peak purity not less than 70% and one or more impurities comprising;
b) oxygenated hemoglobin comprising not more than 2% metHb;
c) oxygenated hemoglobin comprising less than 12% acidic variants of the main peak;
d) oxygenated hemoglobin comprising less than 11% basic variants of the main peak;
e) oxygenated hemoglobin comprising substantially free of virus particles; and
f) oxygenated hemoglobin comprising substantially free of prions.

13. A hemoglobin composition comprising:
a) an oxygenated hemoglobin comprising main peak purity not less than 70%;
b) an acidic variants are less than 12% of the main peak;
c) basic variants are less than 11% of the main peak.

14. The hemoglobin composition according to claim 13, wherein the oxygenated hemoglobin is obtained from heat treated deoxyhemoglobin;
wherein the heat treatment is performed at least for 4 hours.

15. The hemoglobin composition according to claim 12, wherein the acidic variants are less than about 12%, less than about 11%, less than about 10%, less than about 9%, less than about 8%, less than about 7% or less; wherein the basic variants are less than about 11%, less than about 10%, less than about 9%, less than about 8%, less than about 7% or less as analyzed by cIEF (capillary Iso Electric Focusing) and metHb analyzed by co-oximetry.

16. A hemoglobin composition comprising:
a) oxygenated hemoglobin;
b) metHb less than 9% of total oxygenated hemoglobin;
wherein the oxygenated hemoglobin is obtained post heat treatment of a deoxygenated hemoglobin;
wherein the heat treatment is performed for about 10 hours.

17. A hemoglobin composition comprising:
a) oxygenated hemoglobin;
b) metHb less than 4% of total oxygenated hemoglobin;
wherein the oxygenated hemoglobin is obtained post heat treatment of a deoxygenated hemoglobin;
wherein the heat treatment is performed for about 8 hours.

18. The composition according to claim 13, wherein oxygenated hemoglobin has main peak purity about 70%, about 71%, about 72%, about 73%, about 74%, about 75%, about 76%, about 77%, about 78%, about 79%, about 80%, about 81%, about 82%, about 83%, about 84%, about 85% or more.

19. The composition according to claim 16, wherein oxygenated hemoglobin has main peak purity about 70%, about 71%, about 72%, about 73%, about 74%, about 75%, about 76%, about 77%, about 78%, about 79%, about 80%, about 81%, about 82%, about 83%, about 84%, about 85% or more.

20. The composition according to claim 17, wherein oxygenated hemoglobin has main peak purity about 70%, about 71%, about 72%, about 73%, about 74%, about 75%, about 76%, about 77%, about 78%, about 79%, about 80%, about 81%, about 82%, about 83%, about 84%, about 85% or more.

21. The composition according to claim 1, wherein the oxygenated hemoglobin is further conjugated with PEG to form pegylated hemoglobin wherein the pegylated hemoglobin maintains metHb below 5% during storage at 2-8° C. for more than 1 month, 3 months, 6 months, 9 months, 12 months, 15 months, 18 months, 21 months, 24 months, 27 months, 30 months, 33 months, 36 months, 39 months, 42 months, 45 months, and 48 months.

22. The composition according to claim 13, wherein the oxygenated hemoglobin is further conjugated with PEG to form pegylated hemoglobin wherein the pegylated hemoglobin maintains metHb below 5% during storage at 2-8° C. for more than 1 month, 3 months, 6 months, 9 months, 12 months, 15 months, 18 months, 21 months, 24 months, 27 months, 30 months, 33 months, 36 months, 39 months, 42 months, 45 months, and 48 months.

23. The composition according to claim 16, wherein the oxygenated hemoglobin is further conjugated with PEG to form pegylated hemoglobin wherein the pegylated hemoglobin maintains metHb below 5% during storage at 2-8° C. for more than 1 month, 3 months, 6 months, 9 months, 12 months, 15 months, 18 months, 21 months, 24 months, 27 months, 30 months, 33 months, 36 months, 39 months, 42 months, 45 months, and 48 months.

24. The composition according to claim 17, wherein the oxygenated hemoglobin is further conjugated with PEG to form pegylated hemoglobin wherein the pegylated hemoglobin maintains metHb below 5% during storage at 2-8° C. for more than 1 month, 3 months, 6 months, 9 months, 12 months, 15 months, 18 months, 21 months, 24 months, 27 months, 30 months, 33 months, 36 months, 39 months, 42 months, 45 months, and 48 months.

25. The composition according to claim 21 wherein the pegylated hemoglobin maintains metHb below 4% preferably below 3%.

26. The composition according to claim 22 wherein the pegylated hemoglobin maintains metHb below 4% preferably below 3%.

27. The composition according to claim 23 wherein the pegylated hemoglobin maintains metHb below 4% preferably below 3%.

28. The composition according to claim 21 wherein the pegylated hemoglobin maintains:
a) main peak of oxygenated hemoglobin not less than 70%;
b) acidic variants are less than 12% of the main peak;
c) basic variants are less than 11% of the main peak.

29. The composition according to claim 22 wherein the pegylated hemoglobin maintains:
a) main peak of oxygenated hemoglobin not less than 70%;
b) acidic variants are less than 12% of the main peak;
c) basic variants are less than 11% of the main peak.

30. The composition according to claim 23 wherein the pegylated hemoglobin maintains:
a) main peak of oxygenated hemoglobin not less than 70%;
b) acidic variants are less than 12% of the main peak;
c) basic variants are less than 11% of the main peak.
